SEQUENCE LISTING (7 sequences)

SEQ ID NO: 1 - DNA, 2919 bp, Homo sapiens
  CDS (1)..(2919)
atgcggccggagggcgcgggaatggagctcggaggcggcgaggagcgcctgcctgaggagagcaggagggagcactggcagttgctgggtaatttgaagacgacggtggagggtttggtatcaaccaacagccccaacgtctggtctaagtatggtggcttggagcggctttgcagggacatgcagagcatcctctatcacgggcttatccgtgaccaggcgtgccgccgccagacggattactggcagttcgtgaaagacatccggtggctcagtccccactcagcccttcacgtggagaagttcatcagcgtgcacgagaacgaccagagcagtgctgatggtgccagtgaacgtgctgttgccgagctgtggctgcagcacagcctgcagtaccactgcctctcagcccagctccggcccctgctcggggatagacagtatatcagaaaattctacacagatgctgccttcctgctaagtgacgctcatgtcacggccatgctgcagtgcctggaagcagtggaacagaacaacccccgcctcctggctcagatcgatgcgtccatgtttgccagaaagcacgagagcccgctcctggtgacaaagagccagagcctgacagccctgcccagttccacatacacccctccaaacagctatgctcagcattcctactttgggtccttctctagcctccaccaatccgtgcccaacaatggctcagagagaagatctacttcctttccactctctggccctccccggaaacctcaagaaagcagagggcacgtctcaccagcagaggatcaaaccatccaagcccccccagtttcagtctctgcactagccagggattcccctttgaccccaaatgaaatgagctccagtactctgaccagccccatagaggcatcctgggtcagcagccagaatgattccccaggtgatgccagtgaggggcctgagtacctggccattggcaacttggacccccgaggccggactgccagctgtcagagtcacagcagcaatgccgagagcagcagttccaatttgttctcctccagcagctcccagaagccagattctgctgcctcttccttaggggaccaggaaggaggtggggagagccagctgtccagtgtcctccgcaggtccagcttctcagaggggcagacactcactgtcaccagtggggcaaagaaaagccacattcgctcccattcggataccagcattgcctccaggggagctccagaatcctgcaatgataaggcgaagttgagaggccctttgccctactctggtcaaagcagtgaagtcagcacacccagctctctgtacatggaatatgaaggtggtcggtacctgtgctcaggggaaggcatgttccgaagaccatcagaaggacagtccctcatcagctacctctctgagcaagacttcggcagctgtgccgacctggaaaaggagaatgcccacttcagcatctcagagtccttaattgctgccatcgagctaatgaagtgcaacatgatgagccagtgcctagaggaggaggaagtggaagaggaagacagtgatagagagatccaggagctgaagcagaagatccgccttcggcgccagcaaatccgcaccaagaacctgctccccatgtaccaggaggctgagcacggaagctttcgggtcacctccagcagctcccagttcagctcacgtgattcggcacagctctctgactctggctctgctgatgaggttgatgaatttgaaatccaagatgctgacatcagaaggaacacagcctcaagcagcaaatccttcgtttcctcccagtccttctcccactgcttcctgcactccacgtctgctgaggcggtggccatggggctcctgaagcagtttgaggggatgcagcttccagccgcctcggagctggagtggcttgtcccggagcatgatgcccctcagaagctcctgcccattcctgactcactgcccatctcaccggatgacgggcagcacgctgacatctacaagctgcggattcgtgttcgtggcaacttggagtgggccccgccccggcctcagataatttttaatgttcatccagccccaacgaggaaaattgccgtggccaagcagaattaccgctgtgcaggatgtggcatccggactgaccctgattacatcaagcgactgcggtactgtgagtacctgggcaagtacttctgccagtgctgccacgagaatgcccagatggccatccccagccgggttctgcgcaagtgggacttcagcaagtactacgtcagcaacttctccaaggacctgctcattaagatctggaatgatcctctcttcaacgtgcaggacataaacagtgccctctataggaaggtcaagctgctcaatcaagtccggctgctgcgggtccagctgtgtcacatgaagaacatgttcaagacttgccgactggccaaggagcttctggattcctttgacacagtcccaggccacctgacagaggacctccacctgtactcactgaatgacctgactgcgaccaggaagggggagctggggccccggcttgctgagctcaccagggcaggggctacccatgtggagagatgcatgctctgccaagccaaaggcttcatctgtgagttctgtcagaatgaggatgacatcatctttccctttgagctccataagtgccggacctgtgaagagtgtaaagcgtgttaccataaagcctgcttcaagtctggaagctgtccgcgctgcgagcggctgcaggcccggcgggaggcactggccaggcagagcctggagtcttacctgtcagactacgaggaggagcccgcggaagcgctggccctggaagccgccgtcctggaggccacctga

SEQ ID NO: 2 - protein, 972 aa, Homo sapiens
MRPEGAGMELGGGEERLPEESRREHWQLLGNLKTTVEGLVSTNSPNVWSKYGGLERLCRDMQSILYHGLIRDQACRRQTDYWQFVKDIRWLSPHSALHVEKFISVHENDQSSADGASERAVAELWLQHSLQYHCLSAQLRPLLGDRQYIRKFYTDAAFLLSDAHVTAMLQCLEAVEQNNPRLLAQIDASMFARKHESPLLVTKSQSLTALPSSTYTPPNSYAQHSYFGSFSSLHQSVPNNGSERRSTSFPLSGPPRKPQESRGHVSPAEDQTIQAPPVSVSALARDSPLTPNEMSSSTLTSPIEASWVSSQNDSPGDASEGPEYLAIGNLDPRGRTASCQSHSSNAESSSSNLFSSSSSQKPDSAASSLGDQEGGGESQLSSVLRRSSFSEGQTLTVTSGAKKSHIRSHSDTSIASRGAPESCNDKAKLRGPLPYSGQSSEVSTPSSLYMEYEGGRYLCSGEGMFRRPSEGQSLISYLSEQDFGSCADLEKENAHFSISESLIAAIELMKCNMMSQCLEEEEVEEEDSDREIQELKQKIRLRRQQIRTKNLLPMYQEAEHGSFRVTSSSSQFSSRDSAQLSDSGSADEVDEFEIQDADIRRNTASSSKSFVSSQSFSHCFLHSTSAEAVAMGLLKQFEGMQLPAASELEWLVPEHDAPQKLLPIPDSLPISPDDGQHADIYKLRIRVRGNLEWAPPRPQIIFNVHPAPTRKIAVAKQNYRCAGCGIRTDPDYIKRLRYCEYLGKYFCQCCHENAQMAIPSRVLRKWDFSKYYVSNFSKDLLIKIWNDPLFNVQDINSALYRKVKLLNQVRLLRVQLCHMKNMFKTCRLAKELLDSFDTVPGHLTEDLHLYSLNDLTATRKGELGPRLAELTRAGATHVERCMLCQAKGFICEFCQNEDDIIFPFELHKCRTCEECKACYHKACFKSGSCPRCERLQARREALARQSLESYLSDYEEEPAEALALEAAVLEAT

SEQ ID NO: 3 - protein, 956 aa, Mus musculus
MRPEGAGMDLGGGDGERLLEKSRREHWQLLGNLKTTVEGLVSANCPNVWSKYGGLERLCRDMQNILYHGLIHDQVCCRQADYWQFVKDIRWLSPHSALHVEKFISLHESDQSDTDSVSERAVAELWLQHSLQCHCLSAQLRPLLGDRQYIRKFYTETAFLLSDAHVTAMLQCLEAVEQNNPRLLAQIDASMFARKQESPLLVTKSQSLTALPGSTYTPPASYAQHSYFGSSSSLQSMPQSSHSSERRSTSFSLSGPSWQPQEDRECLSPAETQTTPAPLPSDSTLAQDSPLTAQEMSDSTLTSPLEASWVSSQNDSPSDVSEGPEYLAIGNPAPHGRTASCESHSSNGESSSSHLFSSSSSQKLESAASSLGDQEEGRQSQAGSVLRRSSFSEGQTAPVASGTKKSHIRSHSDTNIASRGAAGGPRNITIIVEDPIAEGGQYLCSGEGMFRRPSEGQSLISYLSEQDFGSCADLEKENAHFSISESLIAAIELMKCNMMSQCLEEEEVEEEDSDREIQELKQKIRLRRQQIRTKNLLPAYRETENGSFRVTSSSSQFSSRDSTQLSESGSAEDADDLEIQDADIRRSAVSNGKSSFSQNLSHCFLHSTSAEAVAMGLLKQFEGMQLPAASELEWLVPEHDAPQKLLPIPDSLPISPDDGQHADIYKLRIRVRGNLEWAPPRPQIIFNVHPAPTRKIAVAKQNYRCAGCGIRTDPDYIKRLRYCEYLGKYFCQCCHENAQMVVPSRILRKWDFSKYYVSNFSKDLLLKIWNDPLFNVQDINSALYRKVKLLNQVRLLRVQLYHMKNMFKTCRLAKELLDSFDVVPGHLTEDLHLYSLSDLTATKKGELGPRLAELTRAGAAHVERCMLCQAKGFICEFCQNEEDVIFPFELHKCRTCEECKACYHKTCFKSGRCPRCERLQARRELLAKQSLESYLSDYEEEPTEALALEATVLETT

SEQ ID NO: 4 - protein, 948 aa, Gallus gallus
FLCRKEHWKLLGNLKTTVEGLVSTSNPNVWSKYGGLERLCRDMHSILYHGLIHDKVYCRQKDYWQFVKDIRRLSPSSAHHLEKFISLHENGQPSPDSHSDQAIAQLWLQHSLQFHCLSAQLRPLLGNRQYIRKFYADTAFLLSDAHVTAMLQCLEAVEQNNPRLLAQIDTSMLTRKGESLSSVTKSQSLTALPGSFYTLSPGCMQQHHCGSFSSLPYPASSGLPDRRQASSSISSSSSQPQEHCLASRSSSFSEGRSPLEQPSAVPPSHIPSPKDPFSPANEMSSSTTSQSEDTWTGSQDDPQSDANDGPEYLAIGNLGRRGRACSSASTSSSKSSSSNMFSSSSSQKLDSGDRGANGCGSRGLGLLRRSSFSEGQSSVPQGILKKSHVRSHSDTNVASGKLHESHADTVGGRGTVSSSTQSSELSTPSSLYMEYDSGQYLSSGEGMFRRPSEGQSLISYLSEQDFGSCADLEKENAHFSISESLIAAIELMKCNMMSRQLEEEEEDSDKEIQELKQKIRIRRQQIRTKHLFSTCQEMGSDSLAATDSESQFSSHGSMRLSDSGSADDVEEYEIRDADIKRNPDSSRKPFLSSESISHSFGNSNSAEAVAMGLLKQFEGMQLPAASELEWLVPEHDAPQKLLPIPDSLPVSPDDGEHADIYKLRIRVRGNLEWAPPRPQIIFNVHPAPTRKVAVAKQNYRCAGCGIRTDPDYIKRLRYCEYLGKYFCQCCHENAQTVIPSRILRKWDFSKYYVSNFSKDLLSKIWSDPLFNVQDINPALYRKVKSLNQVWLLRIQLFHMKNMFKTCRLAKDLLDSFDAVPGHLTEDLHLYSLSDLSATKRGDLVPRLTELLKAGSLHVEKCMLCQAKGFICEFCQNEGDIIFPFELNKCKTCEECKACYHKSCFKSTRCPRCERLQARRELLAKQSMESYISDYEDDELEQQEAATAT

SEQ ID NO: 5 - protein, 914 aa, Danio rerio
MEIQEQEDGERRWELWKLLYNLKTTVEGLLSTNNPNVWSRYGGLQRLHKDMNNILGHGLKHEQIYYKQKDYWRFVWCVRYLCPHLARHVEQFSQLEPVLSSSVQSFGEGYKAERWLLHSLQAHVLSAQLKPLLQHKMNTQKYYNDGAFLLSEPHVSAMFQCLEAVEQNNPRMLALIDTVRLSHLKESPLGLMKSQSLCLLPGAPGHCRPADASQSHRLTTSQSCLTEISNSPQSKSSSDVAPSSSAQGRGNQVAGISFTEPVLPESRDFGDGDLDDGPEYLAIGNLGKQKRRDSSSSVHSSEHMGTRGDAHTPLAPPPQRRCSFSEAQKATGRSSRGHIRSLSDTGVTQKQKNESVAATACSKSCGPFSPQSSDSSANSSLYMESNGSQYTSVPDGLFRRPSEGQSLISYLSEQDFGSCADLEKENAHFSISESLIAAIELMKCNMRRPAEEAEEEGDSDSEIQQLKQKIRLRRLQIRRTRLKPAAAASSNQVQSVDSGGSQRSSQDSFHPSDSGSDREVEDIELRDGSDGQSLLAVSQSGLSLSLASLFSDADIKRSASSSRSFLSSESISPSMLQSNSAESVAMGLLRQFEGMQLPAASELDWLVPEHDAPQKLLPIPDSLPISPDDGEHADIYKLRIRVRGNLEWAPPRPQIIFNIHPPPKRKVVIAKQNYRCAGCGIRIEQDYMKRLRYCEYLGRYFCQCCHENAQSVIPAKIIRKWDFSKSYVSNFSRDLLAKISGDPLFNLSDINSALYKKVKALDTVRALREQLNLMKNLLKTCRLAKDVLEEFDSLLPGHLTEDMQLFSLSDLNAVRSGSLTPQLRHLLRLGSTHVTHCMLCQAKGFICEFCGNDKDIIFPYELNKCLRCEDCHACYHKSCFKTGTQCPRCLRLTERRERMARRNMEEQEDEACGT

SEQ ID NO: 6 - protein, 827 aa, Drosophila melanogaster
MTTPPPPPTLASDAIEGAVYAAGEALHHQHDHPQQQQQQQQQQQLARNSLLADLRRSTNFWFRAAKSTDGLQLLAQSCTRILQHGLLQPVELRRLADDFEFLLASAGKQQQQTKPQPYRPDYAARDSASMVNGVEEFLGEWTTRCLRARCLSQCLQTLVSDPELMELYYPSEEAFLRRSGDATALFVCLTAVQLNQSGLLSQLEVRHQVRHRRTCSQPNFSISPKLQAVPEERLQKRIFLRRLKSLPNLSQEDEELDDRAAFRRRCQTFSSSRSHKLREDHLDAPSTSSCSSHDSPRRIQLINCDDIKIWTDQTPQEVPEEVHEKLQQASKASTSVGGYLGSFFGSPPLYTSWFHRGVGDDLNASGDGGMLDTFLPVNGRKLKKQHTLFEGVSMLDEAASSTACISSAPWDIQGSAGDSTTSTTASSSVSITPGSDKMDQQSLASFLQMSRQTHNNTQLEKENAHFRISEACITAIEHVKWSRRLATPAGPTSHNLSEPNLMPYAEQIPGGGMQNHSAEAVGLQLISRFSEQQLPRLGHLKWLVSEQEAPQQLLPLPTPKQDHEQASLTRGTSSWAPPRQQIIFTEHPSESRSKVLQKQNHRCAGCGMRVAKHLQQHFRYCTYLGKYLCTGCHRNQISAIPARILRSWDFRCYPVCSFAYRLIEQMYAFPLFHVPDLNSQLYKHKELAKARKRRLQLQAVKGFIANCRFATREQSFFNAIPAHITQDPDMWSMCDFVDVQNTSMNRSIKEVIALSEQHVHNCVLCTGRAFVCEHCKGGELIYPWQRKVQRCDRCGACFHNGCWKVRSRCRNQNDPCPRCSRLQSRAS

SEQ ID NO: 7 - protein, 597 aa, Caenorhabditis elegans
MFDPSKWFGRAPYQQVGDTDDPIIEVDQISFTSSCSSGEHEGYVTARDTTKNRVNGDSVNDLISFEEDLNLPGTSEENSLLSPDSPSSSTPNFPPQYESQNLKESVIVRNSIFWFTESEVDAAKLVLPISNKGRVCRFDNDVDDNLLETTCRTLQISMGLCERSRWLNHLARDPVMSRLVRELDEDFASTSDEPEDQGAENGEEVSRPPASSFSSSSGSYHTIVGILDETVQLNPLNPGMTRVIVPARRGIQSVDIRRRINETPSAVCSAIIRQAVQKDPPIDQRMMEWMRGFAEVGTWIQEVAPASRKHDKYSYEVPPLSEDWILTIHKQPPIRQGLESQQWKCAACRQSLHTHNINDREQCPRFCDYYGLFFCSLCHGGEKSILPARVLNQWSFTELPVSDRAQRFLRAVRESPVFRIRDLPGDLVKKNKALRAVVELRQKLKHMEGFIKICIDASNQVFEFGNLSTMFASIDRYLLEHDDLFSLNDLQRIYNKFQDLLSLLEPLAKRAREHIIHCKKCRLQAPVCVRCNDMTDRLFAFEERAVSRCEGCGHLSHSPKCPRRDVPRDTHCPKCARLKTRANGGGGRSSRIIGDVV

(19) United States
(12) Patent Application Publication
(10) Pub. No.: US 2021/0308170 A1
(43) Pub. Date: Oct. 7, 2021

(54) COMPOSITION FOR SUPPRESSION OF AGING, PREVENTION, AMELIORATION, OR TREATMENT OF AN AGE-RELATED DISEASE OR SYMPTOM, OR EXTENSION OF LIFESPAN

(71) Applicant: OSAKA UNIVERSITY, Suita-shi, Osaka (JP)

(72) Inventors: Tamotsu YOSHIMORI, Suita-shi, Osaka (JP); Shuhei NAKAMURA, Suita-shi, Osaka (JP)

(73) Assignee: OSAKA UNIVERSITY, Suita-shi, Osaka (JP)

(21) Appl. No.: 17/263,433

(22) PCT Filed: Jul. 26, 2019
(86) PCT No.: PCT/JP2019/029502

§ 371 Date: Jan. 26, 2021

(30) Foreign Application Priority Data
Jul. 27, 2018 (JP) 2018-141252

(51) Int. Cl. A61K 31/7088; A61P 19/10

(52) U.S. Cl. CPC A61K 31/7088; A61P 19/10

(57) ABSTRACT

It was found that Rubicon is involved in aging through suppression of autophagic activity, and it is possible to achieve suppression of aging, prevention, amelioration, or treatment of an age-related disease or symptom, or extension of lifespan, by targeting Rubicon.

### TECHNICAL FIELD

The present invention relates to a composition targeting Rubicon for suppression of aging, prevention, amelioration, or treatment of an age-related disease or symptom, or extension of lifespan. In addition, the present invention relates to a method for evaluating a probability that a test compound has an activity of suppressing aging, an activity of preventing, ameliorating, or treating an age-related disease or symptom, or an activity of extending lifespan. Moreover, the present invention relates to a method for testing a degree of aging, a risk of developing an age-related disease or symptom, or a risk of shortening lifespan, as well as a molecule used for the test.

### BACKGROUND ART

Macroautophagy (hereinafter, referred to simply as “autophagy”) is an evolutionarily conserved intracellular membrane trafficking process in which double-membrane structures called autophagosomes sequester cytoplasmic materials and fuse with lysosomes, where their contents are degraded. Initially, autophagy was described as a bulk degradation system, but it has become clear that autophagy also selectively targets aggregated proteins, lipids, damaged organelles, and invading bacteria. By driving the degradation of a wide range of targets, autophagy maintains cellular homeostasis. Consequently, dysfunction in autophagy has been implicated in many human diseases, including cancer, neurodegeneration, and metabolic disorders.

Recent evidence has shown that autophagy is also involved in animal aging. Autophagic activity decreases with age in many species (NPLs 1 to 3). Studies using C. elegans led to the discovery of several conserved longevity pathways, including mild reduction of insulin/IGF-1 signalling, calorie restriction, germline removal, reduced mitochondrial respiration, and reduced TOR signalling. Importantly, all of these interventions activate autophagy and extend animal lifespan in a manner that depends on active autophagy. This suggests that autophagy is one of convergent mechanisms of many longevity pathways (NPLs 4, 5).

However, the detail of the mechanism by which the relation between the autophagic activity and aging is established has not yet been clarified.

### PRIOR ART DOCUMENT

### NPL

[NPL 1] Chang, J. T. et al., Elife 6, 18459 (2017)

[NPL 2] Uddin, M. N. et al., Age (Dordr) 34, 75-85 (2012)

[NPL 3] Donati, A. et al., J Gerontol A Biol Sci Med Sci 56, B375-383 (2001)

[NPL 4] Toth, M. L. et al. Autophagy 4, 330-338 (2008)

[NPL 5] Madeo, F. et al, J Clin Invest 125, 85-93 (2015)

[NPL 6] Matsunaga, K. et al., Nature cell biology 11, 385-396 (2009)

[NPL 7] Zhong, Y. et al., Nat Cell Biol 11, 468-476 (2009)

### SUMMARY OF INVENTION

### Technical Problem

The present invention has been made in view of the above-described circumstance, and an object of the present invention is to identify a molecule based on which autophagic activity and aging are related. In addition, the present invention aims to achieve suppression of aging, prevention, amelioration, or treatment of an age-related disease or symptom, or extension of lifespan, by targeting the molecule. A further object of the present invention is to test the degree of aging, the risk of developing an age-related disease or symptom, or the risk of shortening lifespan, by targeting the molecule.

### Solution to Problem

Previously, the present inventors identified a negative regulator of autophagy, Rubicon (Run domain Beclin-1 interacting and cysteine-rich containing protein), which inhibits autophagosome-lysosome fusion process (NPLs 6, 7). In view of this, the present inventors investigated whether or not Rubicon is involved in a mechanism based on which autophagic activity and aging are related.

As a result, the present inventors found that the expression of Rubicon increases with age in worm and mouse tissues. This suggests that an age-dependent increase of Rubicon impairs autophagy over time, and thereby curtails animal healthspan. This idea was also supported by the fact that knockdown of Rubicon extended worm and fly lifespan and ameliorated several age-related phenotypes. Tissue-specific experiments revealed that Rubicon knockdown in neurons had the greatest effect on lifespan. Moreover, Rubicon knockout mice exhibited reductions in interstitial fibrosis in kidney and α-synuclein accumulation in the brain, both of which are major age-onset phenotypes. In addition, Rubicon expression was suppressed by several lifespan-extending conditions, including calorie restriction, in both worms and mice.

Moreover, the present inventors found that Rubicon acts downstream of MML-1/Mondo, a convergent transcription factor involved in multiple longevity paradigms. Furthermore, epistatic analysis revealed that two major negative regulators of autophagy, mTOR and Rubicon, regulate lifespan partly independent of each other.

From the above, the present inventors found that Rubicon is a key molecule in aging through suppression of autophagic activity, and it is possible to suppress aging, prevent, ameliorate, or treat an age-related disease or symptom, or extend lifespan, or test the degree of aging, the risk of developing an age-related disease or symptom, or the risk of shortening lifespan, by targeting Rubicon, and completed the present invention.

More specifically, the present invention provides the following:
[1] A composition for suppression of aging, prevention, amelioration, or treatment of an age-related disease or symptom, or extension of lifespan in a subject, the composition comprising a molecule that suppresses functions of a Rubicon gene as an active ingredient.
[2] The composition according to [1], wherein

the molecule that suppresses functions of a Rubicon gene is a molecule described in any one of the following (a) to (c):
(a) an RNA binding to a transcription product of the Rubicon gene or a DNA encoding the RNA,
(b) an RNA or DNA binding to a Rubicon protein, and
(c) an antibody binding to the Rubicon protein.
[3] The composition according to [1] or [2], wherein

the age-related disease or symptom is a metabolic bone disease or a symptom thereof, improvement in stress resistance, or an organ fibrosis.
[4] The composition according to any one of [1] to [3], wherein

the subject is a vertebrate.
[5] A method for evaluating a probability that a test compound has an activity of suppressing aging, an activity of preventing, ameliorating, or treating an age-related disease or symptom, or an activity of extending lifespan in a subject, the method comprising the steps of:

bringing the test compound and a Rubicon protein into contact; and

detecting binding of the test compound and the Rubicon protein, wherein

in a case where the test compound binds to the Rubicon protein, it is evaluated that there is the probability that the test compound has an activity of suppressing aging, an activity of preventing, ameliorating, or treating an age-related disease or symptom, or an activity of extending lifespan.
[6] A method for evaluating a probability that a test compound has an activity of suppressing aging, an activity of preventing, ameliorating, or treating an age-related disease or symptom, or an activity of extending lifespan in a subject, the method comprising the steps of:

bringing the test compound into contact with a cell expressing a Rubicon gene; and

detecting expression of the Rubicon gene in the cell, wherein

in a case where the test compound reduces the expression of the Rubicon gene compared with a case where the test compound is not brought into contact, it is evaluated that there is the probability that the test compound has an activity of suppressing aging, an activity of preventing, ameliorating, or treating an age-related disease or symptom, or an activity of extending lifespan.
[7] A method for evaluating a probability that a test compound has an activity of suppressing aging, an activity of preventing, ameliorating, or treating an age-related disease or symptom, or an activity of extending lifespan in a subject, the method comprising the steps of:

bringing the test compound into contact with a cell having a DNA in which a reporter gene is functionally bound to downstream of a promoter region of a Rubicon gene; and

detecting expression of the reporter gene in the cell, wherein

in a case where the test compound reduces the expression of the reporter gene compared with a case where the test compound is not brought into contact, it is evaluated that there is the probability that the test compound has an activity of suppressing aging, an activity of preventing, ameliorating, or treating an age-related disease or symptom, or an activity of extending lifespan.
[8] A method for testing a degree of aging, a risk of developing an age-related disease or symptom, or a risk of shortening lifespan in a subject, the method comprising the step of:

detecting an expression product of a Rubicon gene in a test sample, wherein

in a case where an amount of the expression product of the Rubicon gene is higher than that of control, it is evaluated that the degree of aging, the risk of developing an age-related disease or symptom, or the risk of shortening lifespan is high.
[9] The method according to any one of [5] to [8], wherein

the age-related disease or symptom is a metabolic bone disease or a symptom thereof, improvement in stress resistance, or an organ fibrosis.
[10] The method according to any one of [5] to [9], wherein the subject is a vertebrate.
[11] A composition for detecting an expression product of a Rubicon gene in the method according to any one of [8] to [10], the composition comprising the following (a) or (b):
(a) an oligonucleotide primer or oligonucleotide probe binding to a transcription product of the Rubicon gene; or
(b) an antibody binding to a Rubicon protein.

### Advantageous Effects of Invention

The present invention makes it possible to achieve suppression of aging, prevention, amelioration, or treatment of an age-related disease or symptom, or extension of lifespan through activation of autophagy by suppressing functions of a Rubicon gene. In particular, it is very surprising that by suppressing functions of a single gene, various advantageous effects can be achieved, such as amelioration of decrease in bone mineral density and bone volume/bone tissue volume fraction, and change in bone morphology, which would cause metabolic bone diseases, reduction of aggregative proteins, which would cause neurodegenerations, reduction of eye degeneration, amelioration of decrease in locomotor function, improvement in stress resistance, reduction of organ fibrogenesis, extension of lifespan, and the like. In addition, the present invention also makes it possible to test the degree of aging, the risk of developing the above-described disease or symptom, or the risk of shortening lifespan by detecting an expression product of the Rubicon gene.

### BRIEF DESCRIPTION OF DRAWINGS

FIG. 1A is diagrams showing that C. elegans Rubicon regulates lifespan via modulating autophagy. (i) shows pictures of anterior intestines at L4 stage. Each RNAi is shown. Autophagosomes (open white arrowheads) labelled with GFP::LGG-1 were more abundant in CeRubicon knockdown, but concomitant knockdown of bec-1/Beclin 1 abolished the increase. The scale bar represents 20 μm. (ii) is a graph of quantification of GFP::LGG-1 puncta in the anterior intestines under each knockdown condition are quantified. Values represent means±s.e.m. (N=3). P-value (**P<0.01) was determined by one-way ANOVA with Tukey's test, relative to control.

FIG. 1B (i) shows representative pictures of intestine in MAH215 (mCherry::GFP::LGG-1) strain subjected to control or CeRubicon RNAi. Autophagic flux is increased by CeRubicon knockdown. Both autophagosome (AP, GFP+mCherry+, indicated by open white arrowheads) and autolysosome (AL, GFP−mCherry+, indicated by open magenta arrowheads) are increased by CeRubicon knockdown. (ii) is a graph in which AP and AL are quantified in each condition. P-value (***P<0.001, ****P<0.0001) was determined by t-test. The scale bar represents 10 μm.

FIG. 1C (i) shows pictures of transgenic worms expressing CeRubicon::EGFP, showing that overall fluorescence is increased at day 7 compared to day 1. The scale bar represents 50 μm. (ii) is a graph of qRT-PCR analysis showing CeRubicon expression at day 1 and day 7 in wild-type worms. Mean±s.e.m. from three independent experiments are depicted and are normalized to wild-type N2 Day-1 samples. P-value (***P<0.001) was determined by t-test.

FIG. 1D Longevity conferred by CeRubicon was abolished by concomitant knockdown of (i) bec-1/Beclin 1 or (ii) unc-51/ULK1. Log-rank test was conducted for statistical analysis.

FIG. 1E (i) is a graph showing a result of qRT-PCR that confirmed that the transgenic worm increased CeRubicon expression. (ii) is a graph showing that overexpression of CeRubicon shortened lifespan of wild-type worms.

FIG. 2A is a diagram showing that whole body Rubicon knockdown ameliorates age-dependent phenotypes. Transgenic worms expressing polyQ35::YFP in body wall muscle exhibited age-dependent accumulation of polyQ inclusions (open white arrowheads) at day 5 stage. CeRubicon knockdown decreased the accumulation of inclusions, and concomitant knockdown of bec-1 reverted the phenotypes. Each RNAi is indicated. The scale bar represents 50 μm.

FIG. 2B (i) is a graph showing the number of polyQ inclusions per worm in FIG. 2A. More than 20 worms were analyzed in each experiment, and the experiments were repeated three times. P-values (*P<0.05, **P<0.01) were determined by t-test. (ii) is a graph showing locomotion speed, calculated from multi-worm tracking analysis at day 5. CeRubicon knockdown worms maintain high locomotion activity. P-value (*P<0.05) was determined by t-test. (iii) is a graph showing that CeRubicon knockdown increased the oxidative stress resistance in an autophagy-dependent manner, and is a graph showing survivorship of wild-type Day-1 worms subjected to indicated RNAi after 4.4 mM H202 treatment for 2 hours. P-value (**P<0.01) was determined by t-test.

FIG. 2C is a graph showing that systemic knockdown of dRubicon significantly extends lifespan of female fly.

FIG. 2D (i) is pictures showing that compound eye degeneration was attenuated by dRubicon knockdown (dRubicon-IR) in flies expressing the expanded polyQ protein MJDtr-Q78s (gmr-GAL4>MJDtr-Q78s flies) compared to control. Open white arrowheads indicate necrotic patches. The scale bar represents 100 μm. (ii) is a graph of quantification of the numbers of necrotic patches in gmr-GAL4>MJDtr-Q78s flies in (i). More than 20 eyes were analyzed for each genotype. P-value (***P<0.001) was determined by t-test.

FIG. 2E (i) is western blotting image showing that Rubicon protein level in kidney was elevated in 20-month-old mice relative to 2-month-old mice. Western blot from WT mice and Rubicon KO (KO) mice shows the specific bands of Rubicon. (ii) is a graph of quantification of western blot shown in (i).

FIG. 2F (i) is western blotting images showing LC-3 and p62 in mice kidney at 20 months of age. (ii) is immunohistochemical images of 20-month-old kidney showing that collagen-I positive area was reduced in Rubicon knock-out (Rubicon KO) kidney relative to WT. P-value (*P<0.05) was determined by t-test. The scale bar represents 100 μm.

FIG. 2G (i) is a graph of quantification of collagen-I positive area in (ii) in FIG. 2F. Values represent means±s.e.m. (WT: N=5; Rubicon KO: N=6). P-value (*P<0.05) was determined by t-test. (ii) is a graph showing a result of qRT-PCR analysis of several fibrosis markers (TGF-b1, Colla1) in 20-month-old kidney. Values represent means±s.e.m. (WT: N=5; Rubicon KO: N=6). P-value (*P<0.05) was determined by t-test.

FIG. 3A is diagrams showing that reduction of Rubicon level in neurons slows down aging. (i) is a graph showing that neuronal CeRubicon knockdown efficiently extended worm lifespan. (ii) is a graph showing that neuronal knockdown of dRubicon extended lifespan in female fly. Transgene expression of dRubicon-IR was induced by the pan-neuronal elav-GAL4 driver.

FIG. 3B (i) is pictures showing that neuronal knockdown of dRubicon1 decreased PolyQ (MJDtrQ78w) inclusions in Kenyon cell layer of female fly brain. The scale bar represents 10 μm. (ii) is a graph of quantification of PolyQ intensity in (i). 10 bilateral layers of Kenyon cell layer from 5 animals were analyzed. P-value (***P<0.001) was determined by t-test. (iii) is a graph showing that dRubicon knockdown in neurons ameliorated the locomotor dysfunction by polyQ expression in female fly. Locomotor function was evaluated by the climbing assay. Data represents mean ±s.e.m. P-values (*P<0.05, **P<0.01, ***P<0.0001) were determined by two-way ANOVA with Tukey's test (elav_MJDtrQ78w vs elav_MJDtrQ78w/Rubicon-IR).

FIG. 3C (i) is pictures showing Lewy body and Lewy neurite-like inclusions containing phosphorylated α-Syn (open arrowheads), 10 months after the injection into mice. These were less abundant in Rubicon knock-out (Rubicon^{flow/flox}:Nestin-Cre) mice than in control (Rubicon^{flow/+}:Nestin-Cre). The scale bar represents 500 μm. (ii) is a graph showing percentages of mice having small (less than 100), moderate (100 to 200), or large (more than 200) numbers of phosphorylated α-Syn positive signals. P-value (*P<0.05) was determined by Chi-squared test (Rubicon^{flow/+}:Nestin-Cre, N=7; Rubicon^{flow/flox}: Nestin-Cre, N=6).

FIG. 4A is diagrams showing that Rubicon is downregulated by several lifespan-extending conditions. (i) is a graph of qRT-PCR analysis showing CeRubicon expression in several long-lived strains including daf-2 (e1370), eat-2(ad465), and glp-1(e2141). Mean ±s.e.m. from three independent experiments are depicted and are normalized to wild-type N2 Day-1 samples. Germline-less phenotype of glp-1 was induced by exposure to elevated temperature (25° C.) for 2 days. P-value (*P<0.05, **P<0.01) was determined by one-way ANOVA with Tukey's test. (ii) is a western blotting image showing that nine-month calorie restriction (CR) decreased Rubicon protein level in kidney compared to ad libitum (AL) feeding.

FIG. 4B (i) is a graph of qRT-PCR analysis showing CeRubicon expression in worms of the indicated strains exposed to the indicated RNAi treatments. Repression of CeRubicon in glp-1germline-less worms was abolished when MML-1 or mxl-2 was knocked down by RNAi. Mean±s.e.m. from three independent experiments are depicted relative to wild-type N2 Day-1 samples. P-value (***P<0.001) was determined by one-way ANOVA with Tukey's test. (ii) is a graph of qRT-PCR analysis showing CeRubicon in wild-type (N2) and MML-1 OE (MML-1 overexpression strain). Mean±s.e.m. from three independent experiments are depicted relative to wild-type N2 Day-1 samples. p-value (*P<0.05) was determined by t-test.

FIG. 4C (i) is a western blotting image showing that MondoA siRNA knockdown significantly increased Rubicon protein level in RPE-1 cells. (Left) of (ii) is a graph showing relative value of Rubicon normalized by Po-S. (Right) of (ii) is a graph of qRT-PCR analysis showing MondoA expression after knockdown with indicated siRNA. The value shows mean±s.e.m. from three independent experiments relative to siLuc control. P-value (****P<0.0001) was determined by t-test.

FIG. 4D is a graph showing that double knockdown of CeRubicon and let-363/mTOR further extended lifespan in comparison with the individual knockdowns.

FIG. 5A is a phylogenetic tree generated by ClustalW analysis showing that Rubicon homolog of C. elegans (Y56A3A.16) is similar to human KIAA00226 (Rubicon), human KIAA0226L, and fruit fly Rubicon (CG12772).

FIG. 5B (i) is representative pictures of pharyngeal muscle in MAH215 (mCherry::GFP::LGG-1) strains subjected to control or CeRubicon RNAi, showing that autophagic flux is increased by CeRubicon knockdown. Both autophagosome (AP, GFP+mCherry+, indicated by open white arrowheads) and autolysosome (AL, GFP−mCherry+, indicated by open magenta arrowheads) are increased by CeRubicon knockdown. The scale bar represents 20 μm. (ii) is a graph of quantification of AP and AL in each condition. P-value (**P<0.01) was determined by t-test.

FIG. 5C (i) is a graph showing that CeRubicon knockdown extended lifespan, but concomitant knockdown of atg-18 abolished the longevity. (ii) is a graph of qRT-PCR analysis showing knockdown efficiency of CeRubicon and atg-18 by RNAi. Mean±s.e.m. from three independent experiments are depicted relative to wild-type N2 with control RNAi.

FIG. 6A is a snapshot of multi-worm tracking analysis showing age-related changes by Rubicon knockdown. Track and speed of individual worms were visualized under the indicated RNAi treatments.

FIG. 6B (i) is a graph showing that pharyngeal pumping rate is not altered in CeRubicon knockdown. (ii) is a graph showing a result of heat stress resistance assay, which revealed that CeRubicon knockdown does not change the resistance.

FIG. 6C (i) is a graph of qRT-PCR analysis showing that dRubicon was significantly downregulated by the knockdown. P-value (****P<0.0001) was determined by t-test (WT: N=3; Rubicon-IR: N=4). (ii) is a graph of demographic analysis of dRubicon knockdown in male fly.

FIG. 6D (i) is pictures and a graph showing a result of the autophagic flux assay in KC layers of brains, which shows that dRubicon knockdown increased autophagosomes (AP, white arrowheads) and autolysosomes (AL, red arrowheads). The values represent the mean±s.e.m. P-values (**P<0.01) were determined by t-test (N=6 for control, N=8 for dRUb-IR1). The scale bar represents 10 μm. (ii) is a western blotting image showing that Rubicon protein levels increased with age (2 months vs 20 months) in mouse liver.

FIG. 7A is diagrams showing contribution of tissue-specific role of Rubicon to lifespan regulation, showing graphs of demographic analysis using several tissue-specific RNAi-sensitive strains such as (i) muscle (NR350), (ii) hypodermis (NR222), and (iii) intestine (VP303). intestinal and hypodermal CeRubicon knockdown slightly but significantly extended lifespan.

FIG. 7B (i) is a graph showing that longevity conferred by neuronal knockdown of CeRubicon is abolished by neuronal atg-18 knockdown. (ii) is a graph of demographic analysis of neuronal dRubicon knockdown in male fly.

FIG. 8A is graphs showing epistatic relationships between CeRubicon knockdown and several longevity pathways. CeRubicon knockdown does not further extend longevity of (i) eat-2 (calorie restriction), (ii) isp-1 (mitochondrial dysfunction), (iii) daf-2 (reduced insulin/IGF-1 signalling), and (iv) glp-1 (germline longevity).

FIG. 8B (i) is a western blotting image showing that 6-month calorie restriction decreases Rubicon protein levels in kidney. (ii) is a graph showing qRT-PCR analysis of CeRubicon expression. Knockdown of MML-1 and mxl-2 do not revert CeRubicon expression to wild-type (N2) level.

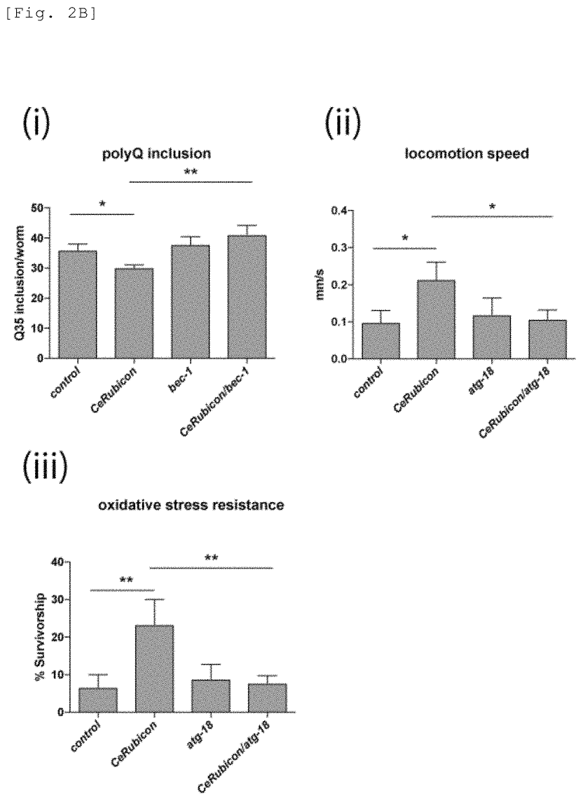

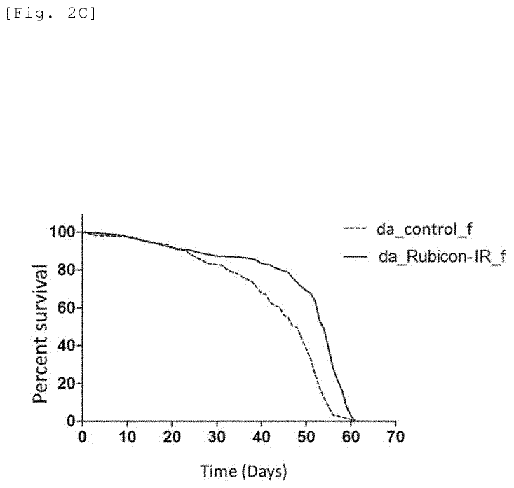

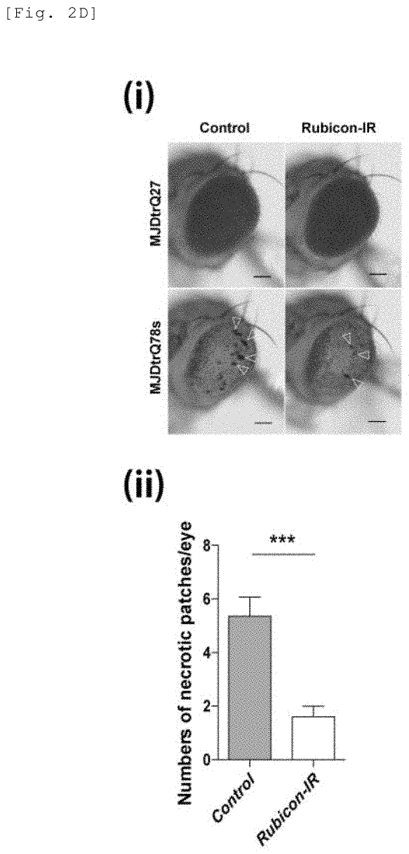

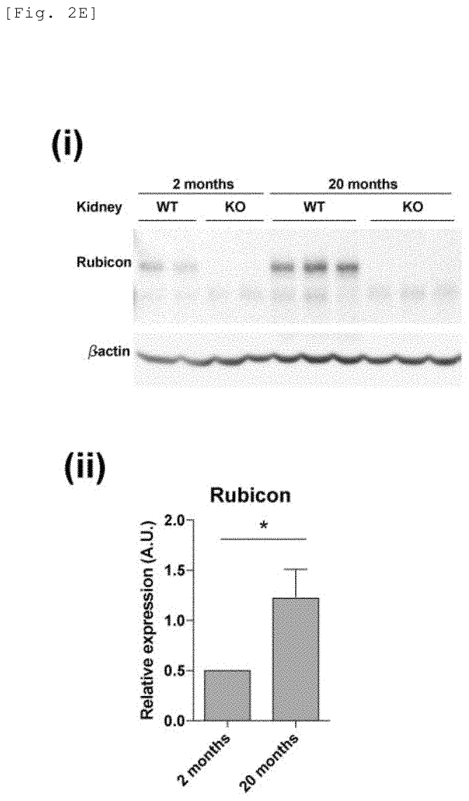

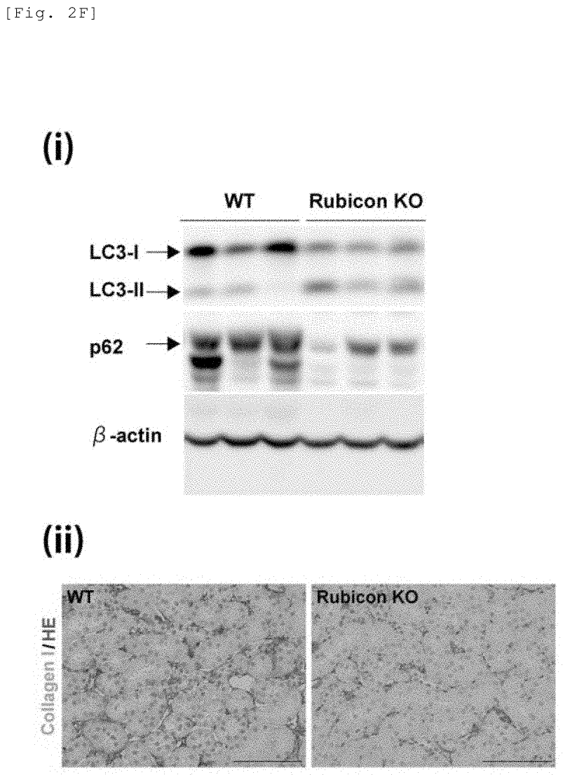

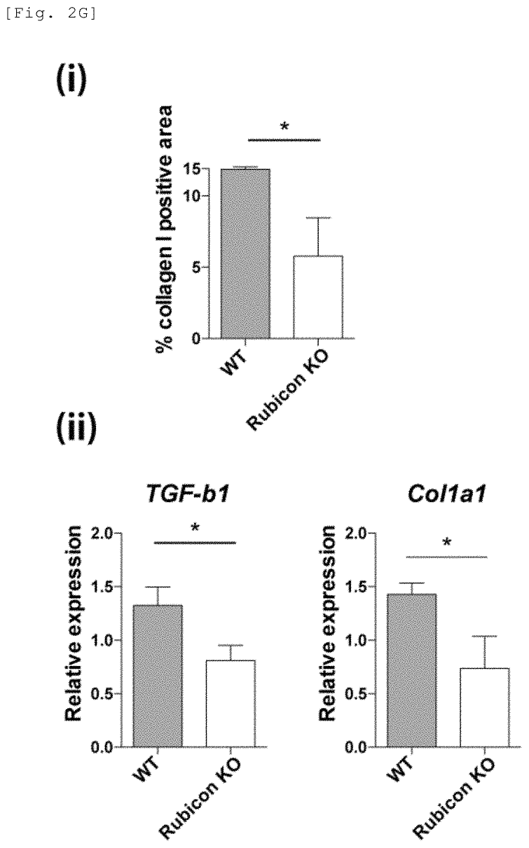

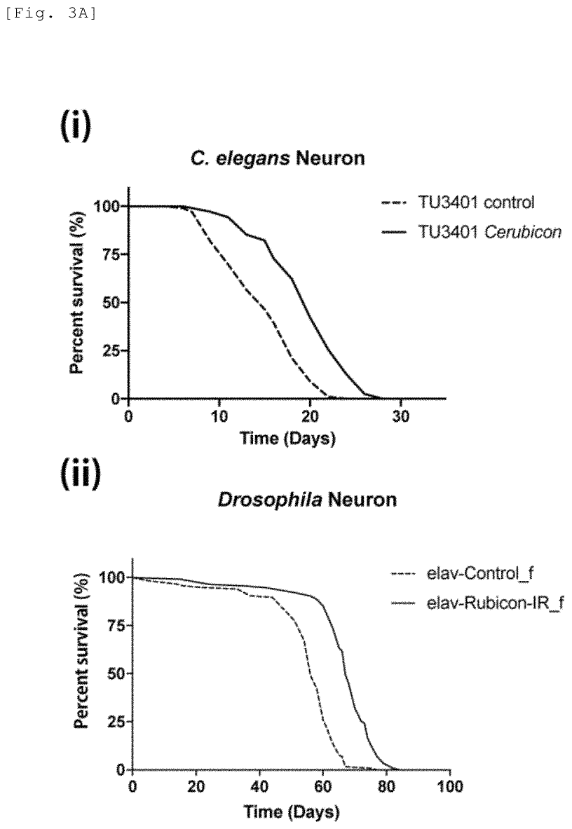

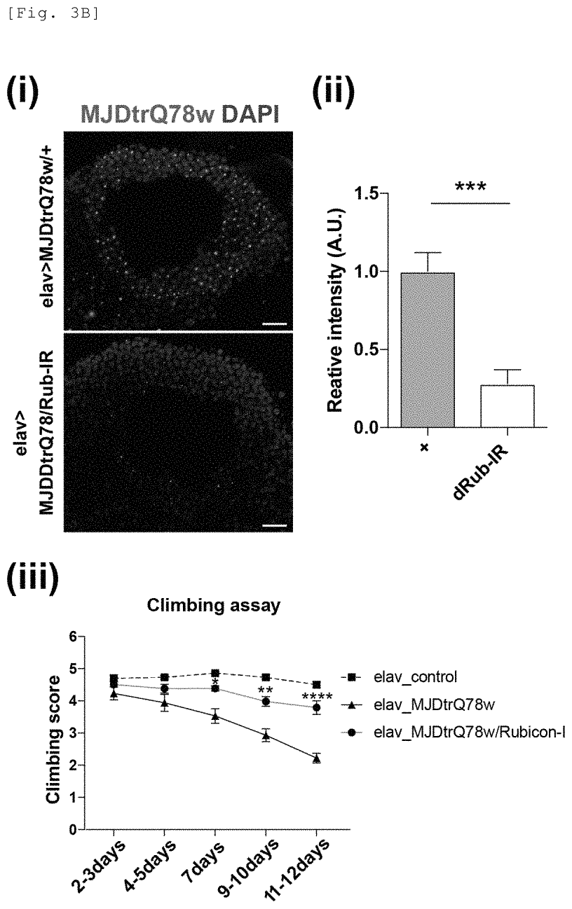

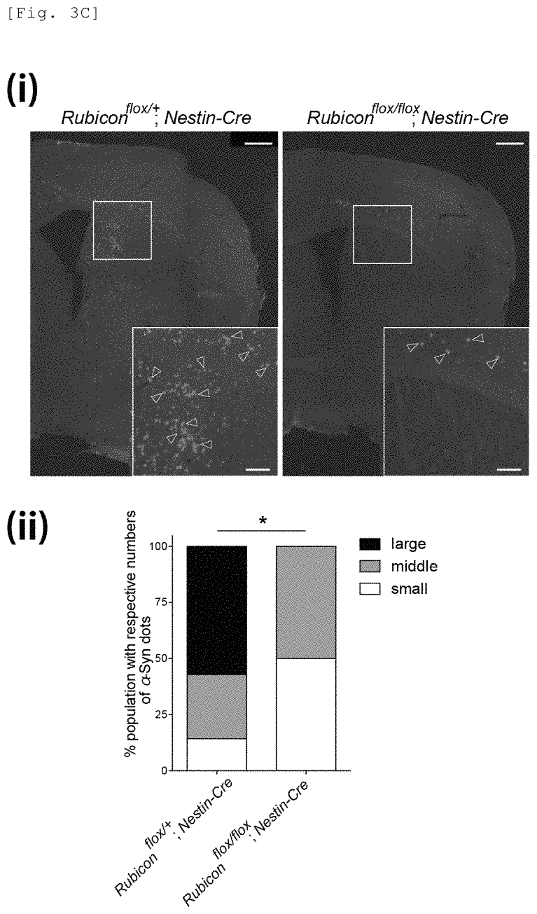

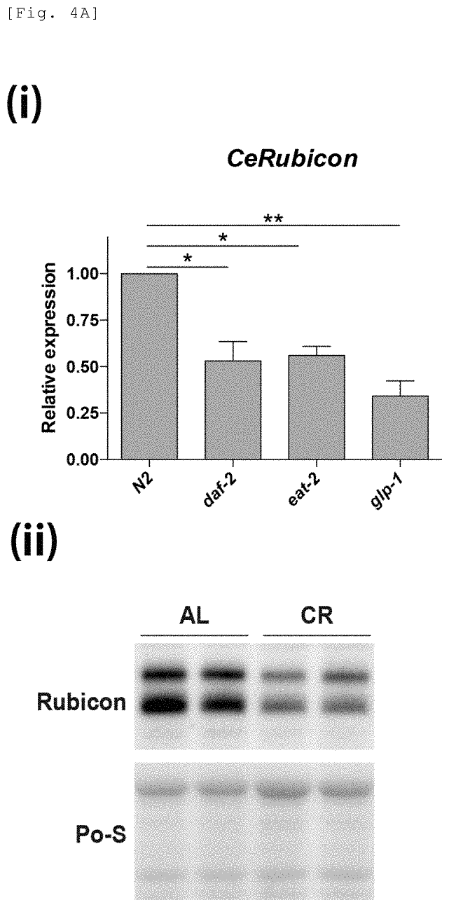

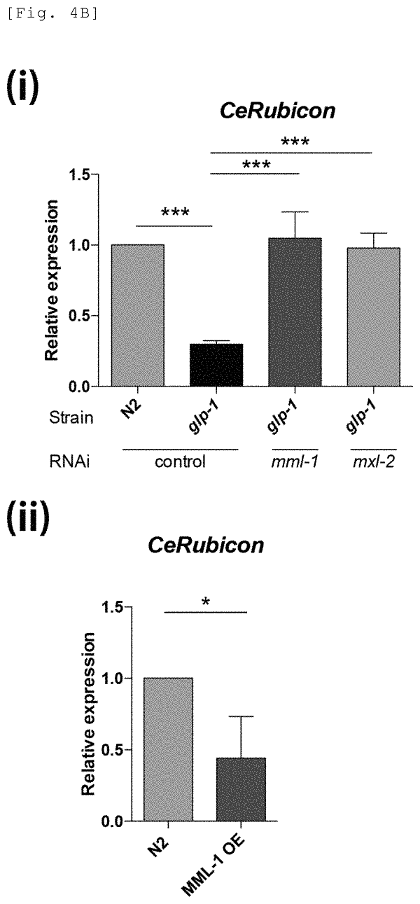

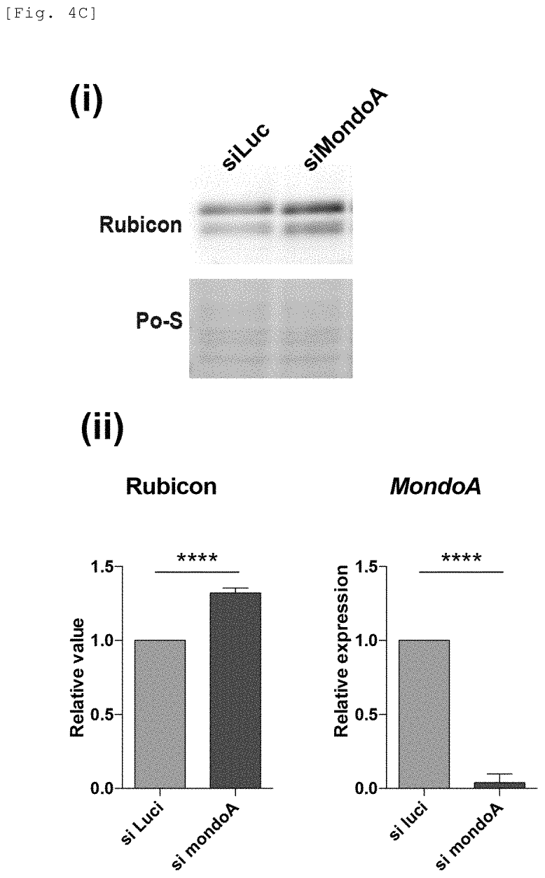

FIG. 2C (i) is a western blotting image showing that knockdown of CeRubicon does not change levels of phosphor S6 kinase, a direct target of mTOR. (ii) is a graph of qRT-PCR analysis of CeRubicon expression showing that CeRubicon is not altered by TOR knockdown.

FIG. 9A (i) is CT images of femurs of Rubicon knockout mice. The scale bar represents 500 μm. (ii) is graphs showing the bone mineral density (BMD), the bone volume/bone tissue volume fraction (BV/TV), and the bone mineral content (BMC) of the femurs of the Rubicon knockout mice. N=6 for wild-type (WT), and N=5 for knockout mouse (KO).

FIG. 9B (i) is pictures in which differentiation of osteoclasts in Rubicon knockout mice was detected through TRAP staining. The scale bar represents 200 μm. (ii) is a graph showing a result of measuring the numbers of TRAP positive cells.

FIG. 9C (i) We evaluated the bone resorption function of osteoclasts in Rubicon knockout mice through pit formation assay. (i) is TRAP-stained images, and (ii) is a graph showing proportions of relative resorption regions in well.

FIG. 9D We evaluated the differentiation potential of osteoblasts in Rubicon knockout mice. (i) is ALP-stained images, and (ii) is graphs showing proportions of ALP-stained areas in well.

FIG. 9E show a result of evaluating mineralization capacity of osteoblasts in Rubicon knockout mice. (i) is alizarin-stained images, and (ii) is graphs showing proportions of alizarin-stained areas in well.

FIG. 10 is a graph and electrophoresis images showing a result of observing facilitation of autophagy in osteoblasts of Rubicon knockout mice through LC3flux assay.

FIG. 11A shows a result of evaluating autophagy-dependency of increase in bone mineral density in Rubicon knockout mice. (i) is pictures showing results of CT-analyzing bone morphology in a Rubicon knockout mouse, an Atg5 knockout mouse, and a Rubicon/Atg5 knockout mouse. (ii) is a graph showing bone densities in the respective mice.

FIG. 11B shows a result of evaluating bone mineral density and bone volume/bone tissue volume fraction in a Rubicon knockout osteoporotic model. (i) is CT images of the entire femurs, (ii) is CT images of trabeculae, and (iii) is graph showing bone mineral density and bone volume/bone tissue volume fraction.

FIG. 12A is a graph showing a result of detecting expression of mRNA in the transcription factor downstream of Notch and the osteogenic transcription factor for evaluating the relationship between autophagy and Notch signal pathway in osteoblasts of Rubicon knockout mice.

FIG. 12B is electrophoresis images and graphs showing a result of detecting degeneration of Notch in osteoblasts of Rubicon knockout mice.

FIG. 13A shows a result of overexpressing NICD in osteoblasts of Rubicon knockout mice and evaluating differentiation potential of the NICD through ALP staining. (i) is ALP-stained images, and (ii) is a graph showing proportions of ALP-stained areas in well.

FIG. 13B is pictures showing a result of detecting binding of NICD and LC3 by the immunoprecipitation method.

### DESCRIPTION OF EMBODIMENTS

<Composition for Suppressing Aging and the Like>

The present invention provides a composition for suppression of aging, prevention, amelioration, or treatment of an age-related disease or symptom, or extension of lifespan in a subject, the composition comprising a molecule that suppresses functions of a Rubicon gene as an active ingredient.

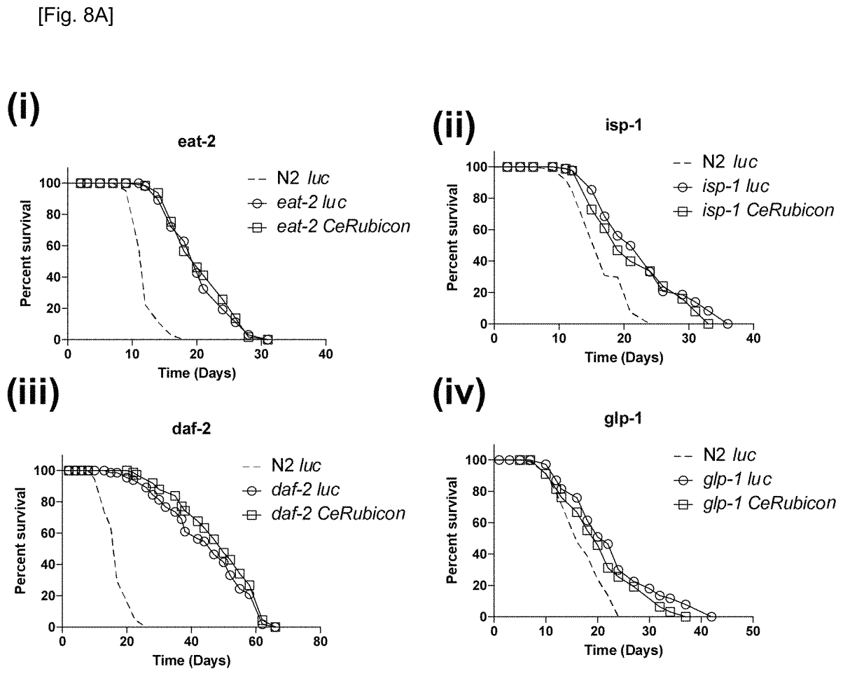

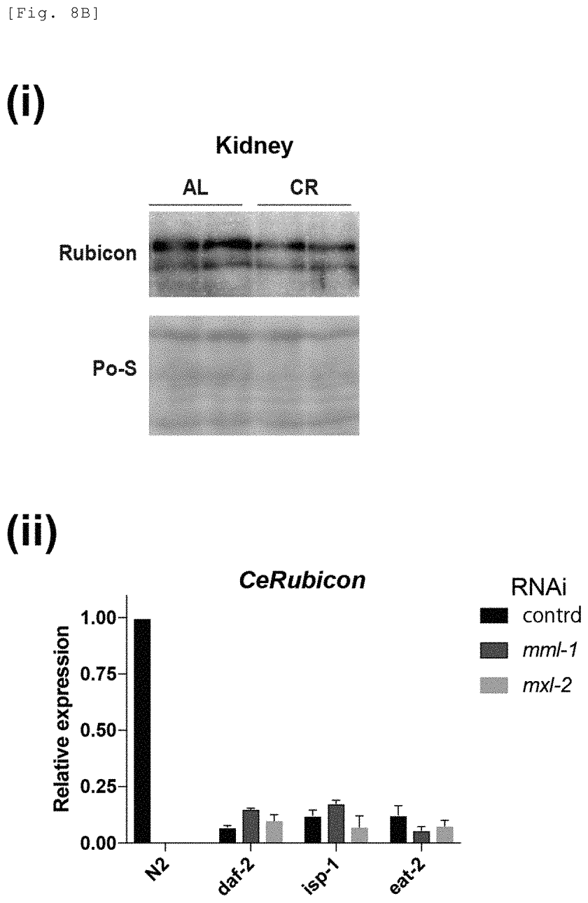

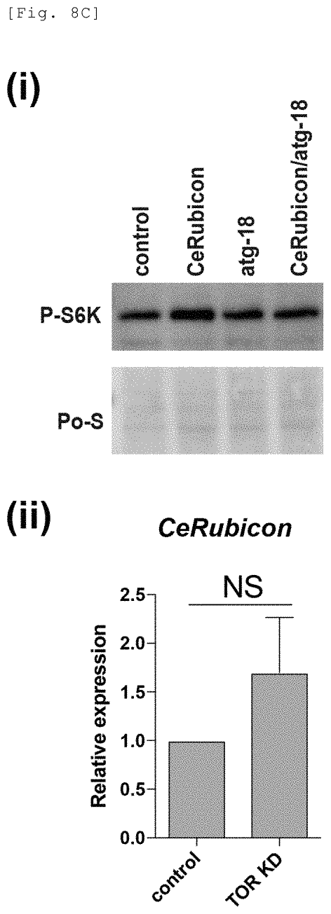

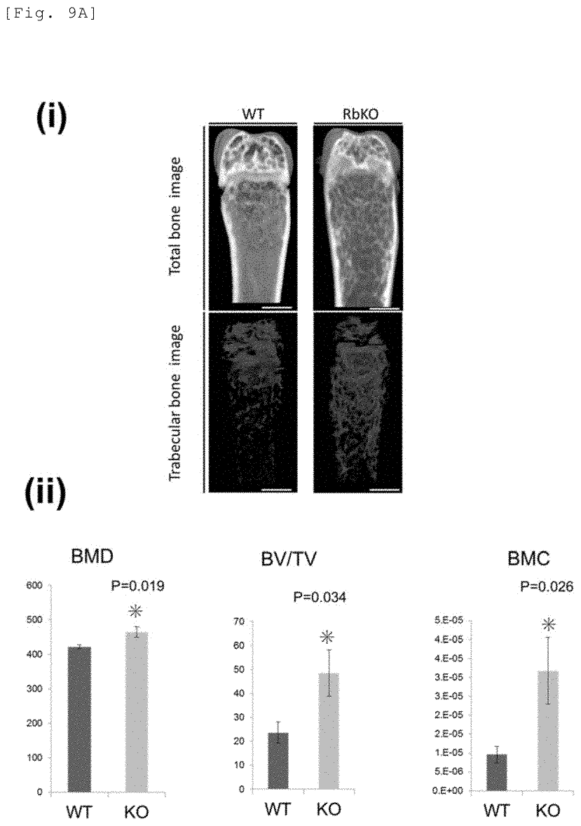

“Rubicon” in the present invention is a negative regulator of autophagy, which inhibits autophagosome-lysosome fusion process (NPLs 6, 7). Rubicon in the present invention may be derived from any living organism (see FIG. 5A). For example, in the case where the composition of the present invention is for humans, Rubicon of the human is targeted. A typical amino acid sequence of the Rubicon protein of the human is shown in SEQ ID NO: 2, and a typical nucleic acid sequence of the cDNA encoding the protein is shown in SEQ ID NO: 1 (database registration No: NM_014687.3). In addition, typical amino acid sequences of the respective Rubicon proteins of the corresponding mouse, chicken, zebrafish, fruit fly, and nematode are shown in SEQ ID NOs: 3 to 7 in this order.

Note that in the sequence of Rubicon, individual differences and mutations may occur. Hence, the Rubicon protein of the present invention includes functionally equivalent proteins formed of amino acid sequences in which one or a plurality of amino acids are substituted, deleted, inserted, or added in the amino acid sequence described in any of SEQ ID NOs: 2 to 7. Here, the “one or a plurality” is normally within 20 amino acids (for example, within 10 amino acids, within 5 amino acids, within 3 amino acids, or 1 amino acid). In addition, “functionally equivalent” means that the protein has the function of negatively regulating autophagy (NPLs 6, 7).

In addition, in comparison (Max score) using BLASTP 2.8.0+(Altschul S. F. et al., Nucleic Acids Res. 25:3389-3402 (1997), Altschul S. F. et al., FEBS J. 272:5101-5109 (2005)) in default settings, the identity (proportion of the same amino acids) and the similarity (proportion of similar amino acids) of Rubicons between different species are respectively 85% and 89% (Gaps 2%) between mouse and human, respectively 73% and 81% (Gaps 2%) between human and chicken, respectively 56% and 66% (Gaps 10%) between chicken and zebrafish, respectively 39% and 57% (Gaps 6%) between zebrafish and fruit fly, and respectively 29% and 47% (Gaps 6%) between fruit fly and nematode. Hence, it is considered that amino acid sequences of Rubicon in species other than the above-described species normally have identity of 25% or more (for example, 30% or more, 35% or more, 40% or more, 45% or more, 50% or more, 55% or more, 60% or more, 65% or more, 70% or more, 75% or more, 80% or more, 85% or more, 90% or more, or 95% or more) and similarity of 45% or more (for example, 50% or more, 55% or more, 60% or more, 65% or more, 70% or more, 75% or more, 80% or more, 85% or more, 90% or more, or 95% or more) with respect to the amino acid sequence of Rubicon in any of the above-described species. Hence, the Rubicon protein of the present invention includes functionally equivalent proteins having amino acid sequences identical or similar to the amino acid sequence described in any of SEQ ID NOs: 2 to 7. Here, “functionally equivalent” means that the protein has the function of negatively regulating autophagy (NPLs 6, 7).

The “aging” in the present invention means a change that occurs in an individual of a living organism along with age. It is possible for individuals to develop various diseases and symptoms with aging. The “age-related disease or symptom” targeted in the present invention is not particularly limited as long as the age-related disease or symptom is caused by the negative regulation of autophagy by Rubicon. The age-related disease or symptom includes, for example, metabolic bone diseases such as osteoporosis, neurodegenerations such as Parkinson's disease, Alzheimer's disease, polyglutamine disease (for example, Huntington's disease, spinocerebellar ataxia), amyotrophic lateral sclerosis (ALS), organ fibrosis (for example, fibrosis of kidney, fibrosis of myocardium), decrease in locomotor function (for example, muscle weakness, endurance weakness, extension of reaction time, decrease in locomotion speed, decrease in motor skill, decrease in deep sensation, decrease in balance ability), decrease in cognitive functions (for example, memory impairment, aphasia, apraxia, agnosia, executive function disorders), decrease in bone mass, bone mineral density and bone volume/bone tissue volume fraction, and change in bone morphology, eye degeneration, decrease in stress resistance, and the like, but is not limited to these. In addition, the “lifespan” in the present invention means a time from when a living organism is born to when the living organism dies. Although lifetime during which a human can maintain the life with his/her own mentality and physicality and live an independent living without depending on daily or continuous medical treatments or cares is particularly referred to as “healthspan”, the “extension of lifespan” in the present invention includes an extension of lifespan achieved by an extension of healthspan.

In fact, although it was confirmed that the lifespan was extended by suppressing the functions of the Rubicon gene in Examples, besides this, the effects of suppressing many age-related diseases and symptoms were also confirmed, such as amelioration of decrease in bone mass, bone mineral density and bone volume/bone tissue volume fraction, and change in bone morphology, which would cause metabolic bone diseases, reduction of aggregative proteins, which would cause neurodegenerations, reduction of eye degeneration, amelioration of decrease in locomotor function, improvement in stress resistances, reduction of organ fibrogenesis, and the like.

As to the “suppression of functions of a gene” in the present invention, the mechanism may be suppression of the expression of the gene (for example, suppression of transcription, suppression of translation) or suppression of the activity of a translation product (protein) of the gene.

The “molecule that suppresses functions of a Rubicon gene” includes, for example, an RNA binding to a transcription product of the Rubicon gene or a DNA encoding the RNA, an RNA or DNA binding to a Rubicon protein, an antibody or low-molecular-weight compound binding to the Rubicon protein, a site-specific nuclease for editing the nucleic acid sequence of the Rubicon gene or DNA of an expression regulating region thereof or the RNA sequence of a transcription product of the Rubicon gene, and the like.

One aspect of the “RNA binding to a transcription product of the Rubicon gene or a DNA encoding the RNA” in the present invention is a dsRNA (double-stranded RNA) complementary to a transcription product of a gene encoding the Rubicon protein or a DNA encoding the dsRNA.

The dsRNA used in the present invention is preferably an siRNA, an shRNA (short hairpin RNA), or an miRNA. The siRNA is a double-stranded RNA formed of a short chain within a range that does not exhibit toxicity in a cell. In addition, the shRNA means a single-stranded RNA in which a sense RNA and an antisense RNA form a spacer sequence in which a hydrogen bond is generated between the sense RNA and the antisense RNA in a cell or the like to cause the spacer sequence to form a hairpin structure and which can be formed into an siRNA by cleavage of the hairpin structure in the cell. Although the miRNA means the form of a single-stranded RNA in general, the form of a double-stranded RNA can be employed as a specific embodiment.

For the dsRNA, the chain length is not particularly limited as long as the dsRNA can suppress the expression of a target gene and does not exhibit toxicity. However, the chain length is generally 15 to 50 base pairs, preferably 15 to 35 base pairs, and further preferably 20 to 25 base pairs. Increasing the chain length makes it possible to enhance the affinity with an mRNA to reduce off-target effects.

The dsRNA or the DNA encoding the dsRNA does not have to be completely identical to the nucleic acid sequence of a target gene, but has a sequence identity of at least 70% or more, preferably 80% or more, and further preferably 90% or more (for example, 95%, 96%, 97%, 98%, 99% or more).

The dsRNA can be prepared by chemically synthesizing the chains, for example, based on the sequence information of the Rubicon gene (for example, SEQ ID NO: 1). The DNA encoding the dsRNA includes an antisense DNA obtained by encoding an antisense RNA corresponding to any region of a transcription product (mRNA) of the target gene and a sense DNA obtained by encoding a sense RNA of any region of the mRNA, and can express the antisense RNA and the sense RNA with the antisense DNA and the sense DNA, respectively. It is also possible to fabricate a dsRNA with these antisense RNA and sense RNA.

The configuration of the case where the expression system of a dsRNA is held in a vector or the like includes a case where an antisense RNA and a sense RNA are expressed from the same vector and a case where an antisense RNA and a sense RNA are expressed from different vectors. In the configuration in which an antisense RNA and a sense RNA are expressed from the same vector, for example, an antisense RNA-expressing cassette and a sense RNA-expressing cassette are constructed by binding promoters capable of expressing short RNAs such as pol III or the like upstream of an antisense DNA and a sense DNA, and these cassettes are inserted into a vector in the same direction or the opposite directions.

In addition, it is also possible to configure an expression system in which an antisense DNA and a sense DNA are reversely arranged to face on different chains. This configuration includes a double-stranded DNA in which an antisense RNA-coding strand and a sense RNA-coding strand are paired, and include promoters facing each other on the opposite sides of the double-stranded DNA such that the antisense RNA and the sense RNA can be expressed from the respective chains. In this case, it is preferable to include terminators at the 3′ terminals of the respective chains (antisense RNA-coding strand and sense RNA-coding strand) in order to avoid addition of extra sequences downstream of the sense RNA and the antisense RNA. In addition, in this expression system of palindrome style, the types of the two promoters are preferably different.

In addition, in the configuration in which an antisense RNA and a sense RNA are expressed from different vectors, for example, an antisense RNA-expressing cassette and a sense RNA-expressing cassette are constructed by binding promoters capable of expressing short RNAs such as pol III or the like upstream of an antisense DNA and a sense DNA, and these cassettes are made to be held by different vectors.

Another aspect of the “RNA binding to a transcription product of the Rubicon gene or a DNA encoding the RNA” in the present invention includes an antisense RNA complementary to a transcription product of the Rubicon gene or a DNA encoding the RNA (antisense DNA).

Antisense RNA or antisense DNA inhibits the process of transcription, splicing, or translation to suppress the expression of a target gene (Hirashima and Inoue, “Shin-Seikagaku Zikken Kouza 2—Kakusan IV—Idenshi No Hukusei To Hatsugen”, edited by The Japanese Biochemical Society, Tokyo Kagaku Dojin Co Ltd, 319-347 (1993)), the antisense RNA or the antisense DNA used in the present invention may suppress the expression of a target gene through any action.

The sequence of the antisense RNA or the antisense DNA is preferably a sequence complementary to a transcription product of Rubicon, but does not have to be completely complementary as long as the antisense RNA or the antisense DNA can effectively inhibit the expression of the gene. The antisense RNA or the antisense DNA has a complementarity of preferably 90% or more (for example, 95%, 96%, 97%, 98%, 99% or more) to a transcription product of the target gene. The chain length of the antisense RNA or the antisense DNA is not particularly limited as long as the antisense RNA or the antisense DNA can suppress the expression of the target gene and does not exhibit toxicity. The length of the antisense RNA or the antisense DNA is generally 10 to 50 bases, and preferably 12 to 30 bases. Although the production cost increases, increasing the chain length makes it possible to enhance the affinity with an mRNA to reduce off-target effects.

The antisense RNA or the antisense DNA can be prepared through chemical synthesis, for example, based on the sequence information of the Rubicon gene. Note that still another aspect of the “RNA binding to a transcription product of the Rubicon gene or a DNA encoding the RNA” in the present invention includes an RNA having a ribozyme activity of specifically cleaving a transcription product of the Rubicon gene or a DNA encoding the RNA. Ribozymes include those having sizes of 400 nucleotides or more such as group I intron-type and M1RNA contained in RNaseP, but also include those having active domains of around 40 nucleotides, which are called hammerhead-type and hairpin-type (Makoto Koizumi and Eiko Otsuka, Tanpakusitsu Kaksusan Kouso, 1990, vol. 35, p 2191). In the present invention, any ribozyme having any form can be used.

The “RNA or DNA binding to a Rubicon protein” in the present invention is typically a nucleic acid aptamer. As the nucleic acid aptamer, DNA is preferable from the viewpoint that DNA has a high stability in living organisms.

The nucleic acid aptamer may contain, as a structural feature, at least one loop structure, a primary structure rich in deoxyguanosine (containing guanosine, guanosine analogue), or deoxyguanosine may have a tetramer cluster structure (so-called “G-quartet structure”). In addition, the nucleic acid aptamer may be either of a double-stranded nucleic acid and a single-stranded nucleic acid, but is preferably a single-stranded nucleic acid.

The length of the nucleic acid aptamer is not particularly limited as long as the number of bases of the nucleic acid aptamer allows the nucleic acid aptamer to specifically bind to the Rubicon protein, but is generally 10 to 100 bases, preferably 15 to 70 bases, and more preferably 20 to 50 bases. Although the production cost increases, a nucleic acid aptamer having a greater chain length can also be used.

The nucleic acid aptamer used in the present invention can be produced by a person skilled in the art selecting a publicly-known method as appropriate. The publicly-known method includes, for example, in vitro selection methods (SELEX method) (Tuerk C. & Gold L., Science. 3; 249(4968): 505-510 (1990), Green R. et al., Methods Compan Methods Enzymol. 2: 75-86 (1991), Gold L. et al., Annu Rev Biochem 64: 763-97 (1995), Uphoff K. W. et al., Curr. Opin. Struct. Biol. 6: 281-288 (1996)).

In the nucleic acid molecule used as an active ingredient of the composition of the present invention, a modified nucleic acid may be used partially or entirely as appropriate from the viewpoint of improving the stability in a living organism, enhancing the affinity with a target (mRNA or protein), or suppressing off-target effects. For example, in an antisense nucleic acid, a gapmer using modified nucleic acids only on the opposite ends can be used as necessary. In a double-stranded RNA as well, for example, a heteroduplex RNA with a gapmer as one chain can be used.

The chemical modification of the nucleic acid includes, for example, modification of a phosphoric acid moiety, modification of a sugar moiety, and modification of a base moiety. Such modification includes, for example, 5-methylation, 5-fluorination, 5-bromination, 5-iodination, and N4-methylation of cytosine; 5-demethylation, 5-fluorination, 5-bromination, and 5-iodination of thymidine; N6-methylation and 8-bromination of adenine; and N2-methylation, 8-bromination, phosphorothioation, methylphosphonation, methylthiophosphonation, chiral-methylphosphonation, phosphorodithioation, phosphoroamidation, 2′-O-methylation, 2′-MOE modification, 2′-AP modification, and 2′-fluorination of guanine, and the like. In addition, modified nucleic acids such as hexitol nucleic acid (HNA), cyclohexene nucleic acid (CeNA), peptide nucleic acid (PNA), glycol nucleic acid (GNA), threose nucleic acid (TNA), morpholino nucleic acid, tricyclo-DNA (tcDNA), 2′-O-methylated nucleic acid, 2′-MOE(2′-O-methoxyethylated) nucleic acid, 2′-AP(2′-O-aminopropylated) nucleic acid, 2′-fluorinated nucleic acid, 2′-F-arabinonucleic acid (2′-F-ANA), and BNA (bridged nucleic acid) can also be used.

BNA includes, for example, α-L-methyleneoxy(4′-CH2-O-2′)BNA or β-D-methyleneoxy(4′-CH2-O-2′)BNA, also called LNA (locked nucleic acid (trade mark), 2′,4′-BNA), ethyleneoxy(4′-(CH2)2-O-2′)BNA, also called ENA, β-D-thio(4′-CH2-S-2′)BNA, aminooxy(4′-CH2-O-N(R3)-2′)BNA, oxyamino(4′-CH2-N(R3)-O-2′)BNA, also called 2′,4′-BNANC, 2′,4′-BNACOC, 3′amino-2′,4′-BNA, and the like.

For the chemical modifications of nucleic acids, see, for example, documents (for example, Satoshi Obika et al., The Journal of the Japanese Pharmacological Society, 148, 100 to 104 (2016), Takao Inoue, Drug Delivery System 31-1, 10 to 22 (2016)) and patent publications (for example, Japanese Patent Application Publication No. Hei 10-304889, International Publication No. 2005/021570, Japanese Patent Application Publication No. Hei 10-195098, Published Japanese Translation of PCT International Application No. 2002-521310, International Publication No. 2007/143315, International Publication No. 2008/043753, International Publication No. 2008/029619, and International Publication No. 2008/049085).

The “antibody binding to a Rubicon protein” used in the present invention may be a polyclonal antibody or a monoclonal antibody, or may be a functional fragment of an antibody. In addition, the “antibody” includes all the classes and subclasses of immunoglobulins. The “functional fragment” of an antibody means a part (partial fragment) of the antibody that specifically recognizes the Rubicon protein. Specifically, the “functional fragment” of an antibody includes Fab, Fab', F(ab')2, variable region fragments (Fv), disulfide bonded Fv, single chain Fv (scFv), sc(Fv)2, diabodies, polyspecific antibody, and polymers of these, and the like.

When the antibody is a polyclonal antibody, the antibody can be obtained by immunizing an immune animal with an antigen (a target protein, a partial peptide of the target protein, or a cell expressing these, or the like), and purifying the antiserum by a conventional approach (for example, salting-out, centrifugation, dialysis, column chromatography, or the like). In addition, the monoclonal antibody can be fabricated by the hybridoma method (Kohler & Milstein, Nature, 256:495 (1975)) or the recombinant DNA method (for example, P. J. Delves, Antibody Production: Essential Techniques, (1997), WILEY, P. Shepherd & C. Dean Monoclonal Antibodies, OXFORD UNIVERSITY PRESS (2000), Vandamme A. M. et al., Eur. J. Biochem. 192:767-775 (1990)).

The antibody binding to a Rubicon protein includes chimeric antibodies, humanized antibodies, and human antibodies. In the case where the antibody is administered to a human as a therapeutic drug, a chimeric antibody, a humanized antibody, or a human antibody is desirable from the viewpoint of reducing side effects.

The “chimeric antibody” is an antibody obtained by binding a variable region of an antibody of a certain species and a constant region of an antibody of a different species. A chimeric antibody can be obtained, for example, by immunizing a mice with an antigen, cleaving an antibody variable domain (variable region), which binds to the antigen, from the gene of the mouse monoclonal antibody, binding the antibody variable domain with an antibody constant domain (constant region) gene derived from a human bone marrow, incorporating the resultant into an expression vector, and introducing the expression vector into a host to produce the chimeric antibody (for example, Japanese Patent Application Publication No. Hei 8-280387, U.S. Pat. Nos. 4,816,397, 4,816,567, ad 5,807,715).

The “humanized antibody” is an antibody obtained by grafting a gene sequence of an antigen-binding site (CDR) of a non-human-derived antibody into a human antibody gene (CDR grafting), and its production method is publicly known(see, for example, EP 239400, EP 125023, WO90/07861, and WO 96/02576).

The “human antibody” is an antibody whose regions are all derived from a human. In the fabrication of a human antibody, it is possible to use a transgenic animal (for example, a mouse) which is capable of producing repertoires of the human antibody through immunization. The method for fabricating a human antibody is publicly known (for example, Jakobovits, A. et al., Nature 362, 255-258 (1993, LONBERG, N. & HUSZAR, D., INTERN. REV. IMMUNOL., 13, 65-93 (1995), Marks J. D. et al., J Mol Biol. 5; 222(3): 581-97 (1991), Mendez M. J. et al., Nat Genet. 15(2): 146-156 (1997), Tomizuka K. et al., Proc Natl Acad Sci U S A. Jan. 18; 97 (2): 722-727 (2000), Japanese Patent Application Publication No. Hei 10-146194, Japanese Patent Application Publication No. Hei 10-155492, Japanese Patent No. 2938569, Japanese Patent Application Publication No. Hei 11-206387, Published Japanese Translation of PCT International Application No. Hei 8-509612, Published Japanese Translation of PCT International Application No. Hei 11-505107).

The “site-specific nuclease for editing the nucleic acid sequence of the Rubicon gene or DNA of an expression regulating region thereof or an RNA sequence of a transcription product of the Rubicon gene” used in the present invention is not limited as long as the site-specific nuclease can be used to site-specifically edit a nucleic acid. However, the CRISPR-Cas systems having Cas proteins as constituent elements are preferable. As the CRISPR-Cas systems, for example, CRISPR-Cas9, CRISPR-Cpf1 (Cas12a), CRISPR-Cas12b, CRISPR-CasX (Cas12e), CRISPR-Cas14, and the like can be used, and besides, as the site-specific nuclease, an artificial nuclease such as TALENs, ZFNs, or PPR (form fused with a base-modifying enzyme) can also be used.

The “low-molecular-weight compound binding to the Rubicon protein” used in the present invention may be a publicly-known compound or a compound identified by an evaluation method or a screening method of the present invention, which will be described later.

The composition of the present invention may be in the form of a pharmaceutical composition, foods and beverages (including animal feeds), or a reagent used for research purposes (for example, in vitro and in vivo experiments).

The pharmaceutical composition may be pharmaceutically formulated using publicly-known pharmaceutical methods. For example, the composition may be formulated into injections, suppositories, capsules, tablets, pills, liquids, powders, granules, subtle granules, film coating agents, pellets, troches, sublingual tablets, masticatory agents, buccal preparations, pastes, syrups, suspending agents, elixirs, emulsions, liniments, ointments, plasters, gel patchs, transdermal therapeutic systems (TTSs), lotions, inhalants, aerosols, or the like to be parenterally or orally used. In these pharmaceutical formulations, pharmacologically acceptable carriers, specifically, sterile water, saline, vegetable oils, solvents, bases, emulsifiers, suspending agents, surfactants, stabilizers, pH adjusters, flavor agents, fragrances, excipients, vehicles, preservatives, binders, diluents, tonicity agents, soothing agents, expanders, collapsing agents, buffers, coating agents, lubricants, coloring agents, sweetening agents, thickening agents, flavoring agents, solubilizers, or other additives, or the like may be incorporated as appropriate. In addition, the pharmaceutical composition may be prepared in the form of liposome delivery system or the like.

The mode of administration of the pharmaceutical composition of the present invention is not particularly limited, and includes, for example, intravenous administration, intraarterial administration, intraperitoneal administration, subcutaneous administration, intradermal administration, tracheobronchial administration, rectal administration, intramuscular administration, administration by transfusion, direct administration (for example, topical administration into affected sites), and the like.

The pharmaceutical composition of the present invention may bind to an auxiliary portion that promotes medically useful properties. Typical useful properties include, for example, promoting the delivery of the compound to the target region (for example, an affected site), maintaining the therapeutic concentration of the compound in the target region, modifying the pharmacokinetic properties and the pharmacodynamic properties of the compound, improving the therapeutic index or safety profile of the compound, and the like.

The molecule for delivery to a target region includes, for example, a brain barrier permeable substance in the case where the target region is the brain. As the brain barrier permeable substance, for example, anti-transferrin receptor antibodies (for example, International Publication No. 2016/208695) and a glycoprotein formed of 29 amino acids derived from rabies virus (see Kumar P. et al., Nature. 2007 5;448 (7149) :39-43 (2007)) are known. Besides, publicly-known molecules for delivery can be used depending on target regions such as the kidney and the liver.

In addition, the pharmaceutical composition of the present invention may be used together with another composition used for suppression of aging, prevention, amelioration, or treatment of an age-related disease or symptom, or extension of lifespan.

In the case where the composition of the present invention is used for foods and beverages, the foods and beverages may be, for example, health foods, functional foods, foods for specified health use, nutrition supplement foods, foods for sick people, food additives, or animal feeds. The foods and beverages can be taken as the compositions described above or can be taken as various foods and beverages. Specific examples of the foods and beverages include products containing oil such as food oils, dressings, mayonnaises, and margarines; liquid foods such as soups, milk beverages, soft drinks, tea beverages, alcohol beverages, nutritious supplement drinks, jelly beverages, and functional beverages; carbohydrate-containing foods such as cereals, noodles, and breads; livestock processed foods such as hams and sausages; marine processed foods such as boiled fish pastes, dried fishes, and salted fish guts; vegetable processed foods such as pickles; semisolid foods such as jellies and yogurts; fermentation foods such as misos and fermented beverages; various confectioneries such as pastries, Japanese sweets, candies, chewing gums, gummy candies, frozen desserts, and ice desserts; retort pouch foods such as curries, foods dressed with thick starchy sauces, and Chinese soups; instant foods such as instant soups and instant miso soups, microwave foods, and the like. Moreover, the foods and beverages include health foods and beverages prepared in the form of powders, granules, tablets, capsules, liquids, pastes, and jellies.

The foods and beverages can be produced in accordance with production technique known in the Technical Field. To the foods and beverages, one or more components effective in suppression of aging, prevention, amelioration, or treatment of an age-related disease or symptom, or extension of lifespan may be added. In addition, the foods and beverages may be combined with a component that exhibits another function or with another functional food to obtain multi-functional foods and beverages.

The composition of the present invention can be used for animals including humans. Animals other than humans are not particularly limited, and the composition may be used for various livestock, poultry, pets, experimental animals, and the like. The animals are preferably vertebrates, include, for example, mammals such as pig, cow, horse, sheep, goat, dog, cat, rabbit, hamster, mouse, rat, and monkey and birds such as chicken, duck, ostrich, and domestic duck, but are not limited to these. For the purpose of researches, the animals may be various experimental experimental living organisms.

In the case of administering or taking the composition of the present invention, the amount of administration or intake can be selected as appropriate depending on the type of the compositions (pharmaceutical products, foods and beverages, or the like), the types, ages, body weights, symptoms, health statuses, and the like of subjects, and the like. In addition, the dosage (in terms of the active ingredient) of the composition of the present invention may vary depending on the type of the active ingredient, but for example, generally 0.1 μg to 10 mg/kg body weight in the case where the active ingredient is an RNA or a DNA, and generally 1 to 10 mg/kg body weight in the case where the active ingredient is an antibody. In addition, the amount of administration or intake per day is also not particularly limited, and may be selected as appropriate in consideration of various factors as described above. In the case where the active ingredient is a DNA encoding the RNA binding to a transcription product of the Rubicon gene, an expression construct may be fabricated and administered such that an appropriate amount of the RNA is expressed.

The present invention also provides a method for suppression of aging, prevention, amelioration, or treatment of an age-related disease or symptom, or extension of lifespan in a subject, including administering the composition of the present invention to the subject or allowing the subject to take the composition of the present invention as described above.

The composition (a pharmaceutical product, foods and beverages, a reagent, or the like) of the present invention or the instruction of the composition may be attached with an indication that the composition is used for suppression of aging, prevention, amelioration, or treatment of an age-related disease or symptom, or extension of lifespan. Here, a “product or an instruction is attached with an indication” means that an indication is attached to the body of the product, a container, a package, or the like, or an indication is attached to an instruction, a package insert, an advertisement, or another printed matter that discloses information on the product. The above-described indication may contain information on the mechanism of action of the composition of the present invention. Such mechanism includes, for example, promotion of autophagy owing to the suppression of functions of the Rubicon gene, and the like.

<Evaluation of Probability that Test Compound has Activity of Suppressing Aging, and the Like>

—Method Using Binding to Rubicon Protein as Index—

As shown in Examples described below, it was revealed that suppressing the functions of the Rubicon gene brought amelioration of various age-related diseases and symptoms, such as amelioration of decrease in bone mass, bone mineral density and bone volume/bone tissue volume fraction, and change in bone morphology, which would cause metabolic bone diseases, reduction of aggregative proteins, which would cause neurodegenerations, reduction of eye degeneration, amelioration of decrease in locomotor function, improvement in stress resistances, reduction of organ fibrogenesis, and the like, and thus extended the lifespan. Hence, as one step for developing the composition for suppression of aging, prevention, amelioration, or treatment of an age-related disease or symptom, or extension of lifespan, it is effective to identify a compound that binds to the Rubicon protein. Specifically, the present invention provides a method for evaluating a probability that a test compound has an activity of suppressing aging, an activity of preventing, ameliorating, or treating an age-related disease or symptom, or an activity of extending lifespan in a subject, the method comprising the steps of: (a) bringing the test compound and a Rubicon protein into contact; and (b) detecting binding of the test compound and the Rubicon protein, as a first aspect of the evaluation method of the present invention.

The “test compound” as a target for the evaluation method of the present invention is not particularly limited, and includes, for example, expression products of gene libraries, synthesized low-molecular-weight compound libraries, peptide libraries, polynucleotide libraries, antibodies, bacteria releasing substances, extracts and culture supernatants of cells (microorganisms, plant cells, and animal cells), purified or partially purified polypeptides, extract sampled from marine living organisms, plants, or animals, and the like. The test compound may be a test compound synthesized based on an in silico design based on the conformation of the Rubicon protein.

The “Rubicon protein” used in the evaluation method of the present invention includes, for example, a protein made of an amino acid sequence described in SEQ ID NO: 2 and partial peptides thereof in the case of a human protein. As the Rubicon protein, besides the above-described natural protein or a partial peptide thereof, an engineered product or a modification product of the protein can be used as necessary. For example, in order to facilitate the detection or purification, a fusion protein with another protein (for example, an enzyme such as alkaline phosphatase (SEAP) and β-galactosidase, a fluorescent protein such as the green fluorescent protein (GFP), a tag such as glutathione-S-transferase (GST)) can be used. The “contact” between the test compound and the Rubicon protein can be achieved by adding the test compound into the evaluation system, or the like. For “detecting binding of the test compound and the Rubicon protein”, a publicly-known method can be employed as appropriate. Such a method includes, for example, a method for identifying a compound that binds to the Rubicon protein by bringing these test compounds into contact with the fixed Rubicon protein. As means for detecting the binding of the test compound and the Rubicon protein, various publicly-known means can be used, but an example of preferable means includes a biosensor utilizing the surface plasmon resonance phenomenon.

In the case where the test compounds are a synthesized low-molecular-weight compound library, for example, the high throughput method utilizing the combinatorial chemistry technology (Wrighton N. C. et al., Science. 26; 273(5274) 458-464 (1996), Verdine, G. L. Nature 384 11-13 (1996), Hogan J. C., Jr Directed combinatorial chemistry. Nature. 384:17-19 (1996)) can be utilized. In the case where the test compounds are a polynucleotide library, for example, the above-described in vitro selection method can be utilized, and in the case where the test compounds are a gene library, for example, a yeast two-hybrid system in the Rubicon protein is expressed as a bait protein to be used.

In a case where the test compound binds to the Rubicon protein As a result of the above-described method, it is evaluated that there is a probability that the test compound has an activity of suppressing aging, an activity of preventing, ameliorating, or treating an age-related disease or symptom, or an activity of extending lifespan.

—Method Using Reduction in Amount of Rubicon Gene Expressed as Index—

As shown in Examples described below, it was suggested that the expression of the Rubicon gene is age-dependently upregulated, which causes the developments of various age-related diseases and symptoms. Hence, it is effective to identify a compound that reduces the amount of the Rubicon gene expressed as one step for developing a composition for suppression of aging, prevention, amelioration, or treatment of an age-related disease or symptom, or extension of lifespan. Specifically, the present invention provides a method for evaluating a probability that a test compound has an activity of suppressing aging, an activity of preventing, ameliorating, or treating an age-related disease or symptom, or an activity of extending lifespan in a subject, the method comprising the steps of: (a) bringing a test compound into contact with a cell that expresses a Rubicon gene; and (b) detecting expression of the Rubicon gene in the cell, as a second aspect of the evaluation method of the present invention.

The “test compound” used in the second aspect is the same as that in the first aspect. For the “Rubicon gene” to be detected, the living organism from which the Rubicon gene is derived is not particularly limited; however the Rubicon gene of humans is preferable in the development of a composition for humans. As the human gene, for example, the gene encoding a protein formed of an amino acid sequence described in SEQ ID NO: 2 (for example, a gene formed of a nucleic acid sequence described in SEQ ID NO: 1) may be detected. In addition, the “cell expressing the Rubicon gene” includes, for example, human cells such as RPE-1 cells, HEK293 cells, and HeLa cells, but is not limited to these. The “contact” of the test compound with the cell expressing the Rubicon gene may be achieved, for example, by adding the test compound into a culture solution of the cells, and the like. For “detecting expression of the Rubicon gene”, a publicly-known method can be used. For “detecting expression of a gene”, the gene may be detected at the transcription level (mRNA level) or may be detected at the translation level (protein level). The method for detecting a gene at the transcription level includes, for example, the RT-PCR, the northern blotting, the in situ hybridization, the dot blotting, the RNase protection assay, and the like. The method for detecting a gene at the translation level includes, for example, the western blotting method, the radioimmunoassay method, the chemiluminescent immunoassay method, the chemiluminescent enzyme immunoassay method, the enzyme immunoassay method, the immunoprecipitation method, the immunochromatography, the immunohistochemical staining method, the imaging cytometry, the flow cytometry, the latex agglutination method, the mass analysis method (MS), and the like.

In a case where the test compound reduces the expression of the Rubicon gene as a result of the above-described method compared with the case where the test compound is not brought into contact, it is evaluated that there is the probability that the test compound has an activity of suppressing aging, an activity of preventing, ameliorating, or treating an age-related disease or symptom, or an activity of extending lifespan.

In the case where the western blotting method is used, for example, reduction in the expression of the Rubicon gene can be evaluated by comparing the amount of the Rubicon protein detected in the presence of the test compound (for example, the band intensity derived from the Rubicon protein) and the amount (control value) of the Rubicon protein detected in the absence of the test compound. Specifically, in a case where the amount of the Rubicon protein in the presence of the test compound is lower than the amount of the Rubicon protein in the absence of the test compound (for example, in the case of 80% or less, 50% or less, 30% or less, or 10% or less of the control value), it can be evaluated that there is a probability that the test compound has an activity of suppressing aging, an activity of preventing, ameliorating, or treating an age-related disease or symptom, or an activity of extending lifespan. In the case of using a method other than the western blotting method in detection of the expression as well, the evaluation can be made using the amount of expression in the absence of the test compound as a control value.

—Reporter System—

The evaluation on whether or not a test compound reduces the expression level of the Rubicon gene can also be conducted with a system using a reporter gene. Specifically, the present invention provides a method for evaluating a probability that a test compound has an activity of suppressing aging, an activity of preventing, ameliorating, or treating an age-related disease or symptom, or an activity of extending lifespan in a subject, the method comprising the steps of: (a) bringing the test compound into contact with a cell having a DNA in which a reporter gene is functionally bound to downstream of a promoter region of a Rubicon gene; and (b) detecting expression of the reporter gene in the cell, as a third aspect of the evaluation method of the present invention.

The “promoter region of a Rubicon gene” is a region that regulates transcription of the Rubicon gene, and means a region upstream of a coding region of the Rubicon gene to which a transcription factor can be bound. A person skilled in the art can obtain the “promoter region of a Rubicon gene”, for example, through screening of a genomic DNA library using the nucleic acid sequence described in SEQ ID NO: 1 or a part thereof as a probe. In the case where a vector in which the reporter gene is bound downstream of an acquired genomic DNA is prepared and introduced into an appropriate cell, if expression of the reporter gene can be derived, the genomic DNA can be identified as the promoter region of the Rubicon gene.

The “reporter gene” is not particularly limited as long as expression of the reporter gene can be detected, and includes, for example, the CAT gene, the lacZ gene, the luciferase gene, the β-glucuronidase gene (GUS), and the GFP gene, which are generally used by a person skilled in the art.

Here, the condition that the reporter gene is “functionally bound” to downstream of the promoter region of the Rubicon gene means that the promoter region of the Rubicon gene and the reporter gene are bound such that expression of the reporter gene is derived by the transcription factor binding to the promoter region of the Rubicon gene.

Note that the “Rubicon gene”, the “test compound”, and the “contact” are the same as those in the second aspect. The “cell” used in the third aspect may be a cell in which once the reporter system is introduced, expression of the reporter gene is derived by the transcription factor binding to the promoter region of the Rubicon gene, and includes, for example, the RPE-1 cell, the HEK293 cell, the HeLa cell, and the like, but is not limited to these.

The expression of the reporter gene can be detected by a method known by a person skilled in the art depending on the type of the reporter gene used. For example, in the case where the reporter gene is the CAT gene, it is possible to detect expression of the reporter gene by detecting acetylation of chloramphenicol attributable to the gene product. It is possible to detect expression of the reporter gene, by detecting color emission of a dye compound due to the catalytic action of the gene expression product in the case where the reporter gene is the lacZ gene, or by detecting chemiluminescence due to the catalytic action of the gene expression product in the case where the reporter gene is the luciferase gene, or by detecting light emission of glucuronic acid or color emission of 5-bromo-4-chloro-3-indolyl-β-glucuronide (X-Gluc) due to the catalytic action of the gene expression product in the case where the reporter gene is the GUS, or by detecting fluorescence by the GFP protein in the case where the reporter gene is the GFP gene.

In a case where the test compound reduces the expression of the reporter gene as a result of the above-described method compared with the case where the test compound is not brought into contact, it is evaluated that there is the probability that the test compound has an activity of suppressing aging, an activity of preventing, ameliorating, or treating an age-related disease or symptom, or an activity of extending lifespan.

In the case where the luciferase gene is used as the reporter gene, for example, reduction in the expression of the reporter gene can be evaluated by comparing the intensity of chemiluminescence detected in the presence of the test compound and the intensity (control value) of chemiluminescence detected in the absence of the test compound. Specifically, in a case where the light emission intensity in the presence of the test compound is lower than the light emission intensity in the absence of the test compound (for example, in the case of 80% or less, 50%or less, 30% or less, or 10% or less of the control value), it can be evaluated that there is a probability that the test compound has an activity of suppressing aging, an activity of preventing, ameliorating, or treating an age-related disease or symptom, or an activity of extending lifespan. In the case of using a reporter gene other than the luciferase gene in detection of the expression as well, the evaluation can be made using the amount of expression detected in the absence of the test compound as a control value.

Although the evaluation method of the present invention has been described so far, it is possible to screen a compound having an activity of suppressing aging, an activity of preventing, ameliorating, or treating an age-related disease or symptom, or an activity of extending lifespan by conducting the evaluation method of the present invention on a plurality of test compounds and selecting a compound using binding to the Rubicon protein, reduction in the expression of the Rubicon gene, or reduction in the expression of the reporter gene as an index. Hence, the present invention also provides such a screening method.

For the compound identified by the evaluation method or the screening method of the present invention, it is preferable to further evaluate the activity of promoting autophagy in cells and verify whether or not the compound actually has the activity in animal experiments and clinical trials. The animal experiments include, for example, amelioration of an age-related disease or symptom in a model animal having the disease or symptom (for example, amelioration of decrease in bone mass, bone mineral density and bone volume/bone tissue volume fraction, and change in bone morphology, which would cause metabolic bone diseases, reduction of aggregative protein, which would cause neurodegenerations, reduction of eye degeneration, amelioration of decrease in locomotor function, improvement in stress resistance, reduction of organ fibrogenesis, and the like), extension of lifespan, or the like, as shown in Examples described below.

<Test for Degree of Aging and the like>

As shown in Examples described below, it was suggested that the expression of the Rubicon gene is age-dependently upregulated, which causes the developments of various age-related diseases and symptoms and shortening of lifespans. Hence, it is possible to test the degree of aging, the risk of developing an age-related disease or symptom, or the risk of shortening lifespan using the amount of expression of the Rubicon gene as an index. Specifically, the present invention provides a method for testing a degree of aging, a risk of developing an age-related disease or symptom, or a risk of shortening lifespan in a subject, the method comprising the step of: detecting an expression product of a Rubicon gene in a test sample, wherein in a case where an amount of the expression product of the Rubicon gene is higher than that of control, it is evaluated that the degree of aging, the risk of developing an age-related disease or symptom, or the risk of shortening lifespan is high. The test method of the present invention can also be expressed as a method for providing information for diagnosis conducted by a doctor or a veterinarian or a method for assisting diagnosis conducted by a doctor or a veterinarian.

The test method of the present invention can be used for animals including humans. Animals other than humans are not particularly limited, and the test method can be used for various livestock, poultry, pets, experimental animals, and the like. Specifically, the animals include mammals such as pig, cow, horse, sheep, goat, dog, cat, rabbit, hamster, mouse, rat, and monkey and birds such as chicken, duck, ostrich, and domestic duck, but are not limited to these. For the purpose of researches, the animals may be various experimental experimental living organisms.

The “test sample” in the test method of the present invention is a biological sample separated from a subject to be tested on the degree of aging, the risk of developing an age-related disease or symptom, or the risk of shortening lifespan. The biological sample is not particularly limited as long as the biological sample contains an expression product (transcription product and/or translation product) of the Rubicon gene, but includes, for example, cells of liver and kidney, and the like.

For the “detection of an expression product of the Rubicon gene”, a publicly-known method can be used. The “detection of an expression product of a gene” may be conducted on a transcription product (mRNA) or on a translation product (protein). The method for detecting a transcription product includes, for example, the RT-PCR, the northern blotting, the in situ hybridization, the dot blotting, the RNase protection assay, and the like. The method for detecting a translation product includes, for example, the western blotting method, the radioimmunoassay method, the chemiluminescent immunoassay method, the chemiluminescent enzyme immunoassay method, the enzyme immunoassay method, the immunoprecipitation method, the immunochromatography, the immunohistochemical staining method, the imaging cytometry, the flow cytometry, the latex agglutination method, the mass analysis method (MS), and the like.

In a case where the amount of the expression product of the Rubicon gene in the test sample is higher than that of the control as a result of detecting the expression product of the Rubicon gene as described above, it is evaluated that the subject has a high degree of aging, a high risk of developing an age-related disease or symptom, or a high risk of shortening lifespan. Here, the “risk of developing” includes not only the risk of development or not but also the risk of earlier development. As the control, the amount of the expression product of the Rubicon gene in a sample separated from an individual that is not aged or an individual that has not developed an age-related disease or symptom can be used, for example.

In addition, the present invention provides a composition for detecting an expression product of a Rubicon gene in the above-described method, the composition comprising the following (a) or (b):
(a) an oligonucleotide primer or oligonucleotide probe binding to a transcription product of the Rubicon gene, or
(b) an antibody binding to a Rubicon protein.

The oligonucleotide primer (hereinafter, referred to simply as “primer”) may be designed such that transcription products other than the transcription product of the Rubicon gene is amplified as little as possible based on the nucleic acid sequence information (for example, SEQ ID NO: 1) of a cDNA encoding Rubicon. A person skilled in the art can conduct such a primer designing by a conventional method. The length of the primer is generally 15 to 50 bases, and preferably 15 to 30 bases, but may be longer depending on the method and purpose.

The oligonucleotide probe (hereinafter, referred to simply as “probe”) may be designed such that the probe binds to transcription products other than the transcription product of the Rubicon gene as little as possible based on the nucleic acid sequence information (for example, SEQ ID NO: 1) of a cDNA encoding Rubicon. A person skilled in the art can conduct such a probe designing by a conventional method. The length of the probe is generally 15 to 200 bases, preferably 15 to 100 bases, and further preferably 15 to 50 bases, but may be longer depending on the method and purpose. In addition, the primer and the probe can be labeled to be used as appropriate.

The primer and the probe can be fabricated, for example, by using a commercially-available oligonucleotide synthesizer. The probe can also be fabricated as a double-stranded DNA fragment obtained through a restriction enzyme treatment or the like. In addition, the primer and the probe may be formed of only natural nucleotides (deoxyribonucleotide (DNA) and ribonucleotide (RNA)) but the above-described chemically modified nucleic acids may be used partially or entirely as necessary. In addition, the primer and the probe can be labeled to be used as appropriate.

As the antibody binding to the Rubicon protein, in the case of detecting the Rubicon protein by the direct method, the antibody to which a labeling substance is bound is generally used. On the other hand, in the case of detecting the Rubicon protein by the indirect method, the Rubicon protein can be detected by using a secondary antibody to which a labeling substance is bound, or the like, without binding a labeling substance to the antibody binding to the Rubicon protein. Here, the “secondary antibody” is an antibody that exhibits reactivity to the antibody binding to the Rubicon protein. For example, in the case where the antibody binding to the Rubicon protein is prepared as a mouse antibody, an anti-mouse IgG antibody can be used as the secondary antibody. Labeled secondary antibodies usable for antibodies derived from various biological species such as rabbit, goat, and mouse are commercially available, and it is possible to select an appropriate secondary antibody for use depending on the biological species from which the antibody binding to the Rubicon protein is derived. In place of secondary antibodies, it is also possible to use protein G or protein A to which a labeling substance is bound.

The labeling substance is not particularly limited as long as the labeling substance can be detected, but includes, for example, radioisotopes such as 125I, 131I, 3H, 14C, 32P, 33P, and 35S, enzymes such as alkaline phosphatase (ALP), horseradish peroxidase (HRP), β galactosidase (β-gal), fluorescent dyes such as fluorescein isothiocyanate (FITC) and rhodamine isothiocyanate (RITC), and fluorescent protein such as allophycocyanin (APC) and phycoerythrin (R-PE), avidin, biotin, metal particles, latex, and the like.

The composition of the present invention may contain other components allowable as reagents such as sterile water, saline, a buffer, and a preservative as necessary besides the above-described molecule as the active ingredient.

In addition, the composition can be combined with other preparations necessary for the test into a kit. The other preparations includes, for example, substrates necessary for detecting labels, positive controls and negative controls, buffers used for diluting and washing samples, and the like. In addition, in the case where an unlabeled antibody is used as a preparation, the kit may contain a preparation obtained by labeling a substance (for example, a secondary antibody, protein G, protein A, or the like) binding to the antibody. Moreover, the kit may contain an instruction manual for the kit.

### EXAMPLES

Hereinafter, the present invention will be described in further detail based on Examples, but the present invention is not limited to Examples described below.

[Materials and Methods]

(1) Growth Conditions and Strains of C. elegans

Nematode was cultured on a nematode growth medium (NGM) agar plate having Escherichia coli strain OP50 at 20° C. using a standard technique. In these Examples, the following worm strains were used.

N2(WT); DA2123, adIs2122[lgg-1p::GFP::lgg-1 +rol-6(su1006)]; AM140, rmIs132 [unc-54p::Q35::YFP]; TU3401, sid-1(pk3321) V; uIs69 [pCFJ90(myo-2p::mCherry) +unc-119p::sid-1]; VP303, rde-1(ne219) V; kbIs7 [nhx-2p::rde-1+rol-6(su1006)]. NR350, rde-1(ne219) V; kzIs20 [hlh-1p::rde-1+sur-5p:NLS:GFP]; NR222, rde-1(ne219) V; kzIs9 [(pKK1260) lin-26p::NLS::GFP+(pKK1253) lin-26p::rde-1+rol-6(su1006)]; CB1370, daf-2(e1370)III; DA465, eat-2(ad465) II; CB4037, glp-1(e2141ts)III; OP198, unc-119(ed3) III; wgIs198 [MML-1::TY1::EGFP::3xFLAG(92C12)+unc-119(+)]; MQ887, isp-1(qm150)IV; MAH215, sqIs11 [lgg-1p::mCherry::GFP::lgg-1+rol-6].

(2) Plasmid Construction and Transgenesis

As a CeRubicon:EGFP translational fusion construct, a coding sequence was cloned into pDC4 vector containing EGFP tag in addition to CeRubicon 4 kb endogenous promoter. The microinjection of the construct was conducted together with co-injection marker myo-2p::mCherry to generate CeRubicon::EGFP.

(3) Mice

CAG-Cre mice and Nestin-Cre mice were obtained from The Jackson Laboratory and Dr. Jun-ichi Miyazaki's Laboratory (Osaka University), respectively. CAG-Cre mice were crossed with Rubiconflow mice (Tanaka, S. et al., Hepatology 64, 1994-2014 (2016)) to produce mice with systemic Rubicon knockout. Resultant mice with the Rubicon-allele were backcrossed into the C57BL/6J wild-type strain five times, followed by intercrossing between Rubicon+/−mice to generate Rubicon−/−mice and wild-type controls. Nestin-Cre mice were crossed with Rubiconflow mice to produce mice having homozygous deletion of Rubicon specifically in the brain. All the mice used in this Example were maintained on a C57BL/6J background with the exception of calorie-restricted mice. For these experiments, an adult onset 40% calorie restriction protocol developed by Turturro et al. (Turturro, A. et al., J Gerontol a-Biol 54, B492-B501 (1999)) was used. Female BDF1 mice were reared individually in cases. The CR protocol was continued until 9 months of age. Water was provided ad libitum.

(4) Fly Stocks and Culture Conditions

Flies were grown on a standard cornmeal-agar-yeast based medium at 25° C.

Flies having UAS-MJDtr-Q27 (#8149), UAS-MJDtr-Q78s (#8150, for eye degeneration), UAS-MJDtr-Q78w (#8141), UAS-GFP-IR (#9330), UAS-dRubicon (CG12772)-IR (#43276), UAS-GFP-mCherry-Atg8a (#37749), da-GAL4 (#55849), and elav-GAL4c155 (#458) were obtained from the Bloomington Stock Center. Transgenic fly lines having gmr-GAL4 have been reported so far (Yamaguchi, M. et al., Oncogene 18, 6767-6775 (1999)).

(5) Individual Number Statistical Analysis

For worms, synchronized eggs were obtained from 4 to 6-hour egg lays on RNAi plates. Starting with day 1 adults, lifespan experiments were set up at a density of 15 to 20 animals per plate, and carried out at 20° C. Worms were transferred to new plates every other day. Survivorship was also counted every other day. All of RNAi lifespan was carried out from egg. Death was recorded as the absence of any movement after stimulation with a platinum wire. Worms that underwent internal hatching or bursting vulva or that crawled off the plates were excluded. For TOR RNAi experiments, knockdown was performed from adults onward. Statistical analysis was conducted on Excel (Microsoft) by using the Mantel-Cox log-rank method. Experimental flies were grown on a standard medium, and were crossed for 48 hours afterbirth, and thereafter, were sorted using CO2 anesthesia. Vials were changed to fresh food without anesthesia every 2 to 3 days, and deaths were recorded until all the flies were died.

(6) RNA Interference

RNA interference(RNAi) was conducted by feeding HT115 (DE3) bacteria transformed with vector L4440, which produces dsRNA against the target gene. Synchronized eggs were placed on the indicated RNAi plate containing IPTG and ampicillin. RNAi clones were obtained from the Ahringer or Vidal RNAi library. let-363/TOR RNAi clones were supplied by Dr. Hansen (Sanford-Burnham Medical Research Institute). In double knockdown experiments, equal volumes of bacteria for respective RNAis are mixed and seeded on plates. Empty vector (L4440) and luciferase (L4440::Luc) RNAi were used as non-targeting controls.

(7) RNA Extraction and qRT-PCR

Tissue samples of worms, flies, and mice were collected in TRIzol (Invitrogen) or QIAZOL (Qiagen). Total RNA was extracted using RNeasy kit (QIAGEN). cDNA was generated using iScript (Bio-Rad) or PrimeScript RT reagent kit (Takara). qRT-PCR was performed with Power SYBR Green (Applied Biosystems) on ViiA7 Real-Time PCR System (Applied Biosystems) or with KAPA SYBR Fast qPCR kit (KAPA Biosystems) on CFX96 Real-Time PCR Detection System (Bio-Rad). Four technical replicates were performed for each reaction. As internal controls, ama-1 (worms), Rp132 (flies), 18s rRNA (mice), and GAPDH (humans) were used.

(8) Multi-worm Tracking Analysis

A 13 cm×10 cm agar-filled plate was divided into four regions having equal area. To keep animals from moving between regions, the regions are surrounded with glycerol, which is an aversive stimulus for C. elegans. Worms from a given experimental group were placed in one of the four regions, and multiple experimental groups were tested simultaneously. An adapted version of the Multi-Worm Tracker (Swierczek, N. A. et al, Nat Methods 8, 592-598 (2011)) was used to record the locomotion of C. elegans on the agar plate. The locomotion was recorded for 10 minutes, and the recording was analyzed using Choreography(part of the Multi-Worm Tracker software) and custom-written scripts to organize and summarize the data. Animal tracks were collected as time series of centroid position for each frame of the final 2 minutes of the recording. The initial 8 minutes were ignored to allow animal recognition by the tracker to stabilize. Choreography filters (shadowless and −t10) were used to avoid image artefacts. The speed of an individual was calculated as the sum of distances between sequential centroid positions, divided by the duration of the track. Experimental groups were summarized using mean weighted by the duration of the animal's track and s.e.m. of the mean.

(9) Climbing Assay

The climbing assay was performed according to a published protocol (Suzuki, M. et al., Human molecular genetics 24, 6675-6686 (2015)) with slight modification. Ten to twenty flies were placed in a conical glass tube (length 15 cm; diameter 2.5 cm) without anesthesia. Ten seconds after tapping the flies to the bottom of the tube, the number of flies in each vertical area was counted and scored as follows: score 0 (0 to 2 cm), 1 (2 to 3.9 cm), 2 (4 to 5.9 cm), 3 (6 to 7.9 cm), 4 (8 to 9.9 cm), 5 (10 to 15 cm). Three trials were performed on each group at intervals of 20 seconds, and the climbing score was calculated as follows: each score multiplied by the number of flies was divided by the total number of flies, and the mean score of the 3 trials was calculated. Results are presented as the mean±s.e.m. of the scores obtained in 3 to 9 independent experiments.

(10) Use of Microscopy and Quantification

To monitor autophagic activity, GFP:LGG-1 and mCherry:GFP:LGG-1 animals at the L4 stage were anesthetized in 0.1% sodium azide, and images were acquired using an Olympus FV1000 confocal microscope. Using these images, GFP and/or mCherry puncta in the anterior intestinal region or in the pharyngeal region were counted and quantified. For CeRubicon:EGFP and Q35::YFP worms, fluorescent images of the aligned whole worms on ice cold empty plates were captured by stereomicroscope SZX16 equipped with DP80 CCD camera (Olympus) and the number of polyQ aggregates per worms was counted.

(11) Imaging of Fly Eyes

Light microscopic images of 1-day-old adult female flies were taken using a stereomicroscope model SZX16 (Olympus) equipped with CCD camera (DP22, Olympus). The number of necrotic patches per compound eye was counted for each genotype.

(12) Autophagic Flux of Flies

To match the feeding status, 4-day-old flies expressing GFP-mCherry-Atg8a either with or without dRubicon-IR under the da-GAL4 driver were starved by 0.75% agar for 15 hours and refed for 4 hours. The brains were dissected, fixed in 4% formalin in PBS, incubated with 80% glycerol overnight, and then mounted with DAPI fluoromount-G (SouthernBiotech). Fluorescent images of the KC layers were taken by confocal microscope (Leica TCS SP8) and the number of GFP and/or mCherry puncta in 25×50 pm region of interest containing 150 to 200 cells were counted. Three un-overlapping images were used for quantification of each KC layer.

(13) Stress Resistance Assay

Day-1 adult worms were subjected to oxidative stress (4.4 mM H₂O₂ in the empty plate) or heat stress (35° C.) for 3 hours or 7 hours, respectively, and the survived worms were counted. 30 worms were used per condition and the experiments was repeated three times

(14) Western Blotting

Worm or mouse tissues were lysed in lysis buffer (50 mM Tris/HCl [pH 7.4], 150 mM NaCl, 1 mM EDTA, 0.1% NP-40, protease and phosphatase inhibitor cocktail [Roche]) using a homogenizer. After centrifugation, the resultant supernatants were subjected to protein quantification and western blotting. Protein lysates of worm and mouse tissues were separated by SDS-polyacrylamide gel electrophoresis and transferred to PVDF membranes, which were then blocked and incubated with specific primary antibodies. Primary antibodies and dilutions used for mouse tissues western blotting were as follows:

Rubicon (Cell Signalling Technology, #8465, 1:500), LC3 (Cell Signalling Technology, #2755, 1:1000), p62 (MEDICAL & BIOLOGICAL LABORATORIES CO., LTD., PM045, 1:1000), phospho-p70 S6 kinase (Thr398) (Cell Signalling, #9209, 1:500), and β-actin (Sigma Aldrich, A5316, 1, 8000).

(15) Immunohistochemistry

For flies, adult female brains (3 days after eclosion) were dissected, fixed in 4% formalin in PBS, blocked with 50% Block Ace (Sumitomo Dainippon Pharma Co., Ltd.) in PBS/T (0.5% Triton X-100 in PBS) and incubated with an anti-HA antibody (3F10, 1:500, Sigma) to stain MJDtrQ78 protein with HA tag. The immunostaining were visualized with an Alexa 488-conjugated anti-rat antibody and nuclei were stained with DAPI. Images of Kenyon cell layer (KC layer) were taken by confocal microscope (LSM780, Zeiss). Signal intensities of HA staining were quantified using Image J 1.51 s software. Ten bilateral KC layers from five animals were analyzed for each group. For mice, immunohistochemical staining for collagen-I was performed on paraffin-embedded section. After antigen retrieval by autoclaving in 0.01 mmol/L citrate buffer (pH 6.0) for 10 minutes at 120° C., the sections were blocked with 3% BSA in PBS for 60 minutes. The blocked sections were incubated with primary antibodies, anti-collagen-I (abcam, ab34710, 1:400) at 4° C. overnight, incubated for 30 minutes at room temperature, followed by detection using a HRP-diaminobenzidine compound (Nichirei Corp.). The sections were counterstained with hematoxylin.

(16) Injection and Detection of Recombinant α-Syn Fibrils

Injection of fibrils and their detection were reported previously (Luk, K. C. et al., Science 338, 949-953 (2012), Watanabe, Y. et al., Autophagy 13, 133-148 (2017)).

In brief, prepared fibrils were diluted in sterile PBS and sonicated before intracerebral injection. mice between 8 to 10 weeks of age were anesthetized with chloral hydrate (250 mg/kg, i.p.) and stereotaxically injected in both both hemispheres (coordinates: +0.2 mm relative to bregma, +2.0 mm from midline) with recombinant α-Syn fibril(1 μl of 1 mg/ml). Animals were killed at 10 months after birth. Frozen brain sections including the fibril-injected areas were obtained using CM3050S cryostat (Leica Biosystems). The sections were incubated with mouse monoclonal antibody pSyn#64 (dilution, 1:1000, Wako Pure Chemical Industries, Ltd.), followed by Alexa Fluor 488 labeled secondary antibody (dilution, 1:1000, Invitrogen) for detection. Images were acquired and quantified on Biorevo BZ-9000 (Keyence Corporation).

(17) Cell Culturing

RPE-1 cells were inoculated at a density of 5.0×104 cells per 6 wells. After one day, cells were treated with 20 nM siRNA relative to luciferase and MondoA (Santa Cruz). After the siRNA treatment over 48 hours, the RPE1 cells were lysed in a sample buffer and subjected to SDS-polyacrylamide gel electrophoresis and western blotting. The Rubicon protein levels were quantified by western blotting. One day before the siRNA treatment, RPE-1 cells were inoculated at a density of 5.0×104 per 6 wells.

(18) Statistical Analysis

Results are presented as the mean±s.e.m. Statistical tests were performed through one-way or two-way ANOVA with Tukey's test or the t-test using GraphPad Prism (GraphPad Software or Excel (Microsoft Office 2011).

(19) TRAP Staining

Bone marrow cells were cultured into a 24-well plate and derived to osteoclasts by adding M-CSF and RANKL. The resultant was fixed with 4% paraformaldehyde for 5 minutes, and after washing with distilled water, 50 mM tartaric acid-containing buffer (Ph 5.0) and chromogenic substrate were added, followed by reaction at 37° C. for 45 minutes. The stain by TRAP activity was microscopically observed with BZ-X700 (Keyence).

(20) Pit Formation Assay

were cultured into an osteo assay plate (Corning #3988), and derived to osteoclasts by adding M-CSF and RANKL. Osteoclasts dissolved the bone substrate to form resorption lacunae (Pit), which were microscopically observed with BZ-X700 (Keyence).

(21) ALP Staining

Cells were cultured for 5 days in a bone differentiation medium with 12-well plate for the primary culture system and 96-well plate for the culture cell system. The bone differentiation medium was prepared with αMEM supplemented with 10% FBS, 50μg/ml L-ascorbate phosphate (Sigma), 10 mM β-glycerophosphate (Sigma). The resultants were fixed with 4% paraformaldehyde (in 0.1M cacodylate buffer; pH 7.3) for 30 minutes, and Bromo-Chloro-Indolyl phosphate/Nitro Blue Tetrazolium Chloride was added to stain at 37° C. for 20 minutes, which were then microscopically observed with BZ-X700 (Keyence).

(22) Alizarin Staining

Cells were cultured for 4 weeks in a bone differentiation medium with 12-well plate for the primary culture system and 96-well plate for the culture cell system. The bone differentiation medium was prepared with αMEM supplemented with 10% FBS, 50μg/m1 L-ascorbate phosphate (Sigma), 10 mM β-glycerophosphate (Sigma). The bone differentiation medium was replaced every two days, and the resultant was fixed with 4% paraformaldehyde (in 0.1M cacodylate buffer; pH 7.3) for 30 minutes. The resultant was washed with 0.1M cacodylate buffer (pH 7.3) and stained with Alizarin Red S (pH 4.0) for 5 minutes, followed by washing again with distilled water, which were then dried and microscopically observed with BZ-X700 (Keyencne).

(23) Bone Analysis

C57BL/6 background male mice of 12 weeks of age were used for analysis. Note that in osteoporotic models, ovaries were extirpated from female mice of 9 weeks of age, and bone morphology was measured after 4 weeks. For bone morphology, the distal ends of the femurs of mice were CT-scanned (3D micro-X-ray CT for small and medium experimental animals/CosmoScan AX; Rigaku). Thereafter, the 3D micro-CT data were analyzed with TRI/3D-BON software (RATOC Systems) to analyze the bone morphology.

### EXAMPLES

(1) Regulation of Lifespan through Adjustment of Autophagy by Rubicon

We searched for C. elegans Rubicon homologs and found that Y56A3A.16 has sequence similarity to human Rubicon (KIAA0226), human KIAA0226L (recently identified as Pacer) (Cheng, X. et al., Mol Cell 65, 1029-1043 (2017)) and the fruit fly Rubicon (dRubicon, CG12772) (Banreti, A. et al., Dev Cell 28, 56-69 (2014)) (FIG. 5A).

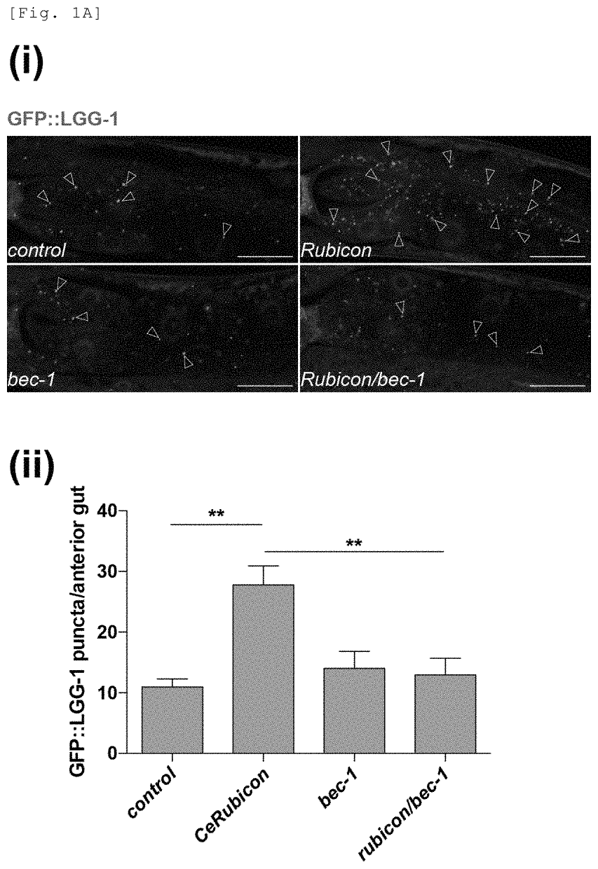

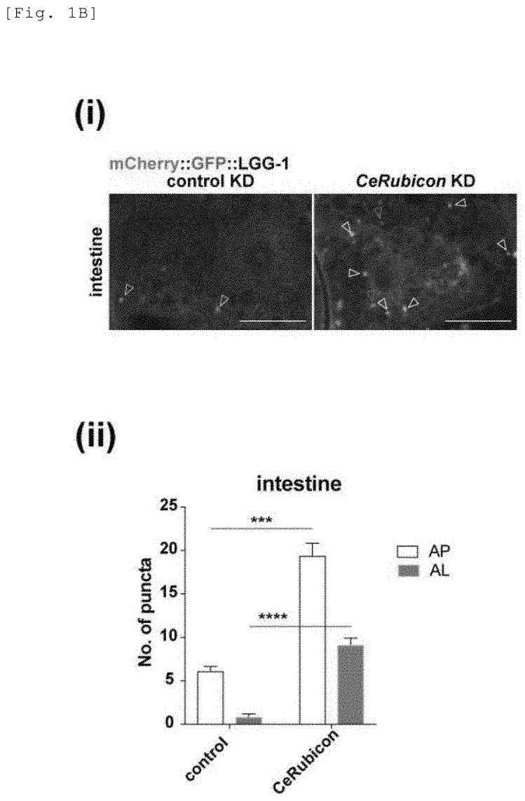

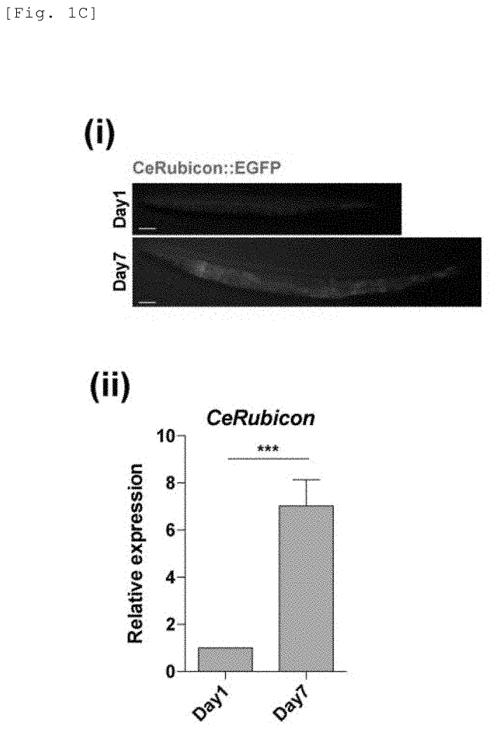

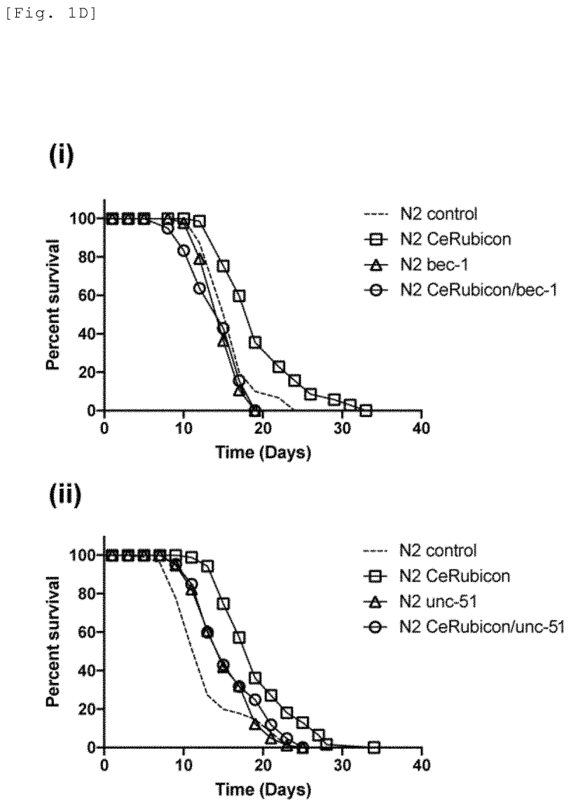

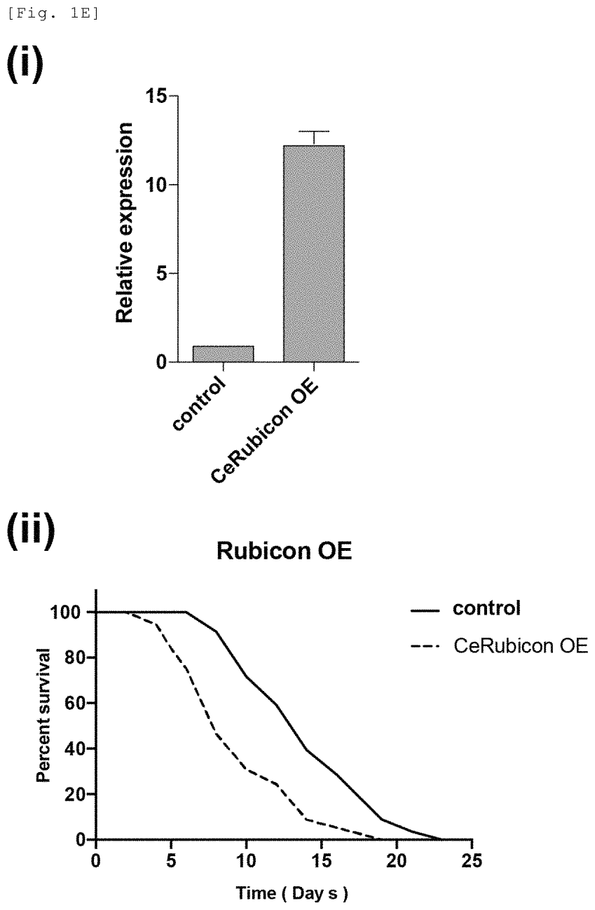

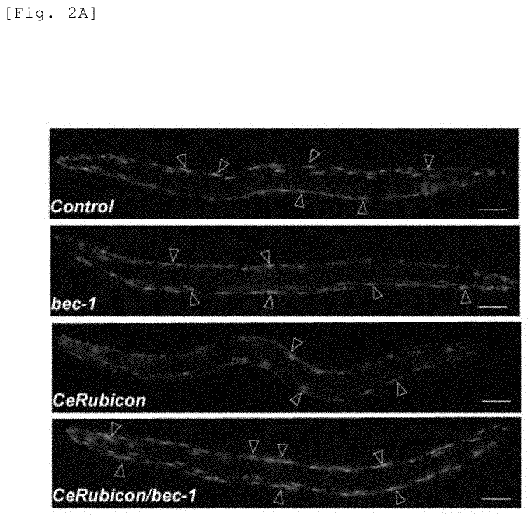

In mammals, Rubicon inhibits autophagy, but Pacer is reportedly required for normal progression of autophagy. Therefore, we next, sought to determine which of these two proteins is more functionally similar to Y56A3A.16. As observed in Rubicon knockdown mammalian cells (NPL 6), knockdown of Y56A3A.16 by RNAi significantly increased the number of GFP:LGG-1 dots, which are an autophagosome marker, in enterocytes ((i), (ii) in FIG. 1A). This increase was completely abolished by concomitant knockdown of bec-1/Beclin 1, both of which function in the autophagosome formation. This fact suggests that knockdown of Y56A3A.16 increased autophagic vacuoles ((i), (ii) in FIG. 1A).

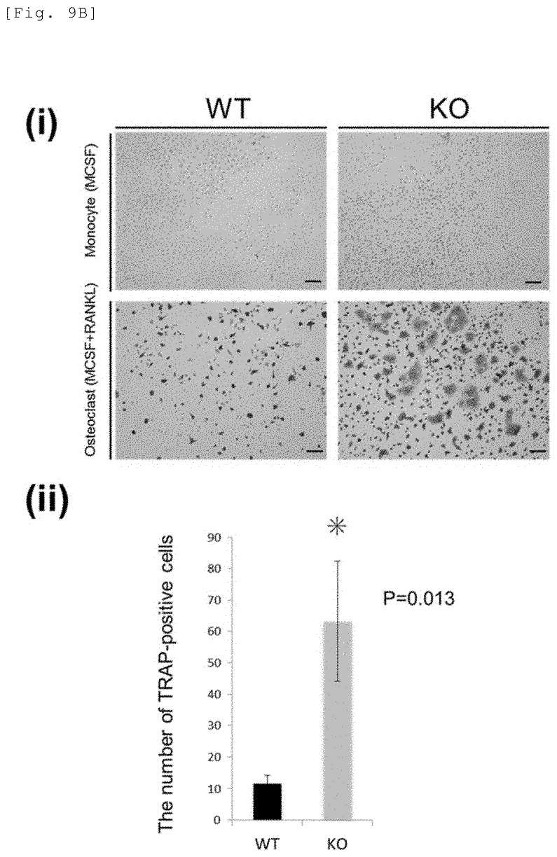

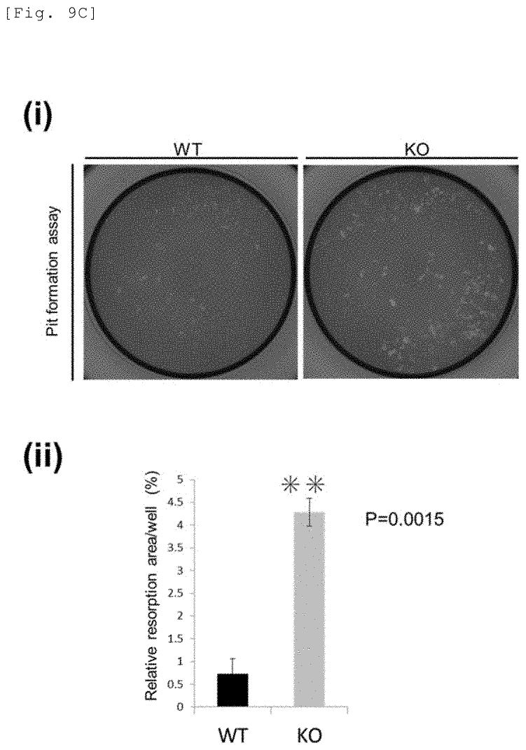

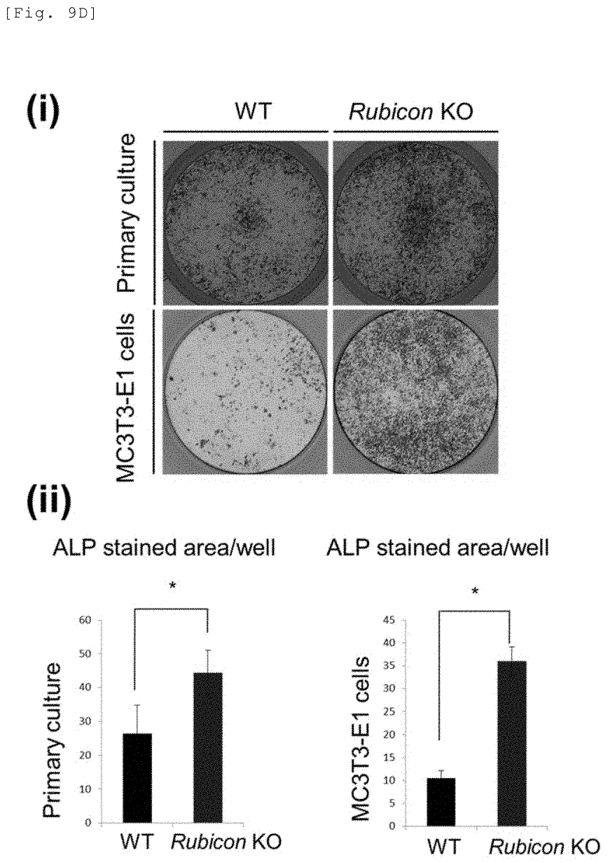

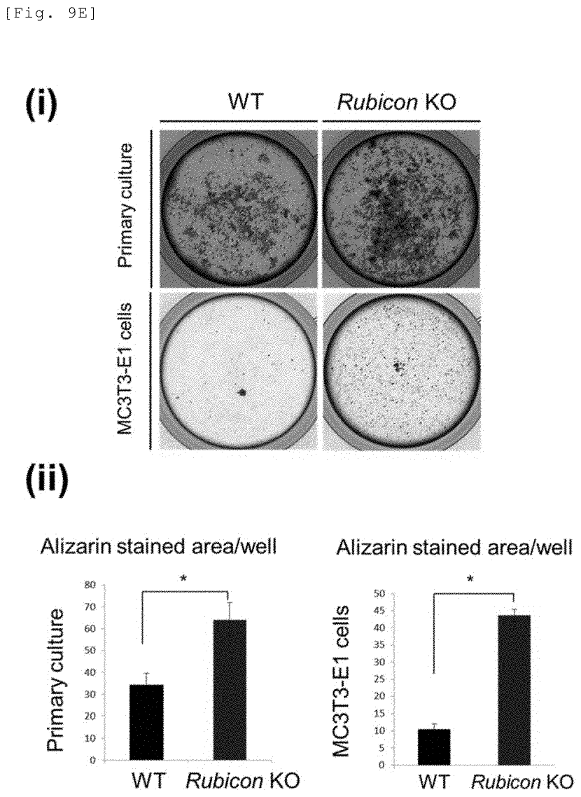

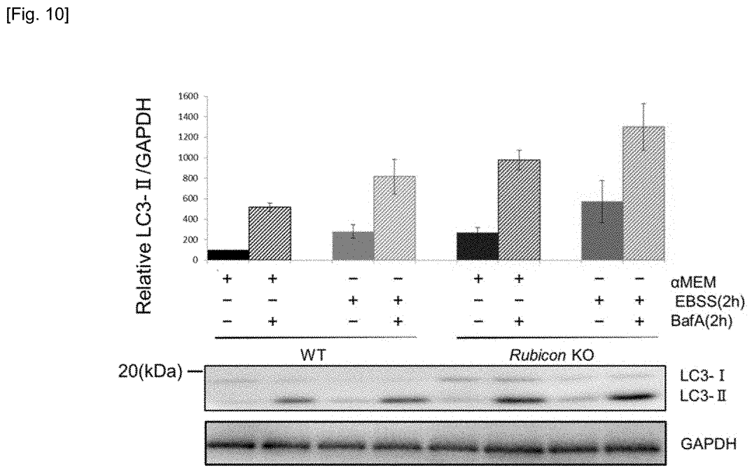

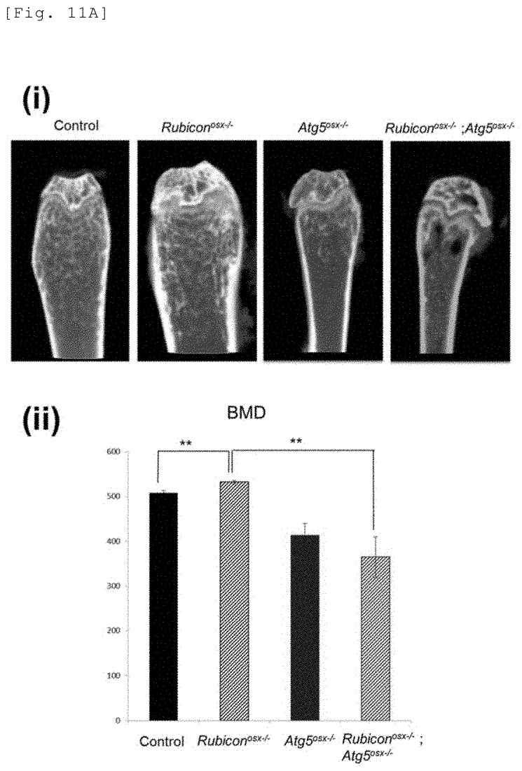

To further determine whether knockdown of Y56A3A.16 increases or blocks the autophagic activity, we used the recently developed transgenic worms expressing tandem fluorescent LGG-1 (mCherry:GFP:LGG-1) to monitor autophagic flux (NPL 1). We observed that knockdown of Y56A3A.16 significantly increases the numbers of autophagosomes (AP) and autolysosomes (AL) in intestines and pharyngeal muscles ((i), (ii) in FIG. 1B, (i), (ii) in FIG. 5B). This indicates the autophagic flux is enhanced by the knockdown. Taken together with the fact that there is only one Rubicon homolog in zebrafish and fruit fly (FIG. 5A) (Banreti, A. et al., Dev Cell 28, 56-69 (2014)), this result implies that Y56A3A.16 represents the ancestral homolog of Rubicon (hence, Y56A3A.16 is referred to as “CeRubicon”). Interestingly, we found that gene expression experiments using CeRubicon:EGFP transgenic worms and qRT-PCR analysis revealed that CeRubicon was upregulated in old worms (day 7) in comparison with young worms (day 1) ((i), (ii) in FIG. 1C). These results led us to hypothesis that age-dependent increase of Rubicon, a negative regulator of autophagy, could be a causal factor for age-dependent impairment of autophagy. Consistent with this idea, CeRubicon knockdown by RNAi significantly extended wild-type worm lifespan ((i), (ii) in FIG. 1D, (i) in FIG. 5C). The CeRubicon knockdown does not alter the pharyngeal pumping rates, indicating that the bacteria containing RNAi are properly taken in ((i) in FIG. 6B). The transcription level of Rubicon was decreased to about 50% of the normal levels of these animals ((ii) in FIG. 5C).

In mammalian cells, Rubicon has been shown to repress both autophagy and endocytosis, but an increase in lifespan was completely abolished when the autophagy regulators bec-1/Beclin 1, unc-51/ULK1, and atg-18/ATG 18 were knocked down by RNAi with CeRubicon ((i), (ii) in FIG. 1D, (i) in FIG. 5C). This indicates that the longevity enhancement conferred by the CeRubicon knockdown is dependent on autophagic activity. Conversely, we found that the overexpression of CeRubicon shortened the lifespan of wild-type worms ((i), (ii) in FIG. 1E).

These results as described above suggest that CeRubicon regulates the lifespans by modulating autophagic activities.

(2) Improvement of Phenotypes Depending on the Age by Systemic Rubicon Knockdown

We investigated whether the knockdown of Rubicon would impact other age-onset phenotypes. Worms expressing polyglutamine (polyQ)-YFP in body wall muscles exhibit age-onset aggregation of the polyQ fusion protein (Morley, J. F. et al., P Natl Acad Sci USA 99, 10417-104229 (2002)). Although the CeRubicon knockdown reduced the level of aggregation, the concomitant knockdown of bec-1/Beclin 1 reverted the reduction. This indicates that the CeRubicon knockdown reduces aggregation of polyQ fusion protein in an autophagy-dependent manner (FIG. 2A, (i) in FIG. 2B). Locomotion activity goes down with age, and velocity correlates well with longevity (Hahm, J. H. et al., Nature Communications 6, doi:ARTN 8919). A multi-worm tracking system allowed us to monitor the tracks of individual worms and calculate their locomotion speeds (Swierczek, N. A. et al., Nat Methods 8, 592-598 (2011)). This analysis revealed that the CeRubicon knockdown slowed down the age-dependent decline in locomotion activity in an autophagy-dependent manner ((ii) in FIG. 2B, FIG. 6A).

Many long-lived animals often show the resistances against several stresses including oxidative stresses and heat stresses. We found that the CeRubicon knockdown increases the oxidative stress resistance in an autophagy-dependent manner ((iii) in FIG. 2B). On the other hand, the heat stress resistance did not change ((ii) in FIG. 6B). This indicates that the CeRubicon knockdown confers different downstream outputs with regard to the stress resistances.

To extend the findings in C. elegans, we asked if the Rubicon knockdown also extends the lifespans of flies. As a result, the systemic knockdown of fruit fly Rubicon (dRubicon) significantly increased the autophagic flux in the brain and extended the lifespan in female-specific manner (FIGS. 2C, (i), (ii) in FIG. 6C, (i) in FIG. 6D). Notably, the knockdown of dRubicon also improved compound eye degeneration in MJDtr-Q78 (a truncated form of MJD/SCA3 containing an expanded length glutamine tract) polyQ disease model female flies ((i), (ii) in FIG. 2D). Rapamycin, an inhibitor in mTOR kinase treatment, increases more in female mice than in male mice. Thus, this observation might reflect the sexual dimorphic contribution of autophagy to the animal lifespan. Similar to C. elegans, we found that the Rubicon protein level in kidney and liver was higher in older mice (20-month old) than in younger mice (2-month old) ((i), (ii) in FIG. 2E, (ii) in FIG. 6D). To understand the role of Rubicon regarding animal aging in mice, we developed Rubicon systemic-knockout mice which exhibit higher levels of LC3-II and reduced levels of autophagy substrate p62, suggesting activation of basal autophagy ((i) in FIG. 2F). Progressive fibrosis is a histological hallmark of aging kidney (Yamamoto, T. et al., Autophagy 12, 801-813 (2016)). Rubicon systemic-knockout mice exhibited a reduction in age-related fibrosis, as determined by the immunohistochemistry of collagen-I ((ii) in FIG. 2F, (i) in FIG. 2G). This phenotype was confirmed by qRT-PCR detection of mRNA encoding fibrotic markers ((ii) in FIG. 2G). In summary, knockdown or deletion of Rubicon ameliorated age-related phenotypes in worms, flies, and mice.

(3) Slowing Down Aging Due to Decrease in Rubicon Levels in Neurons

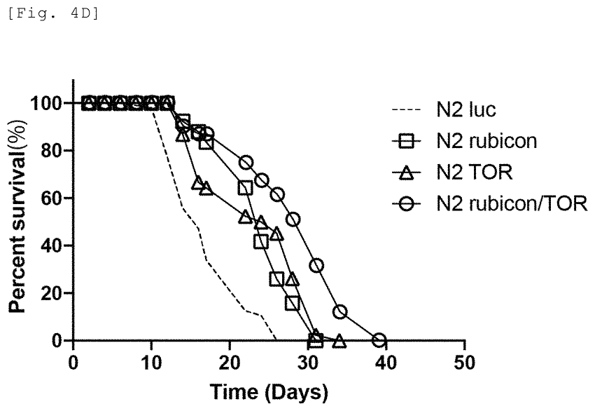

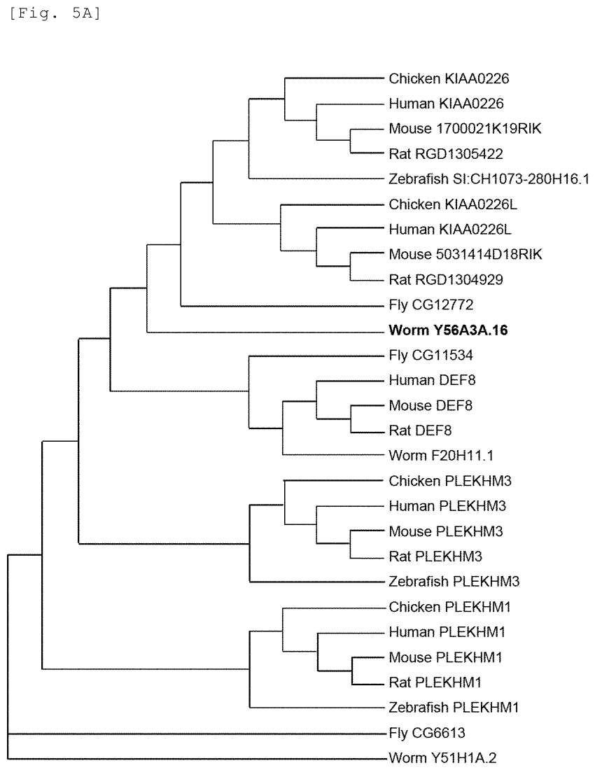

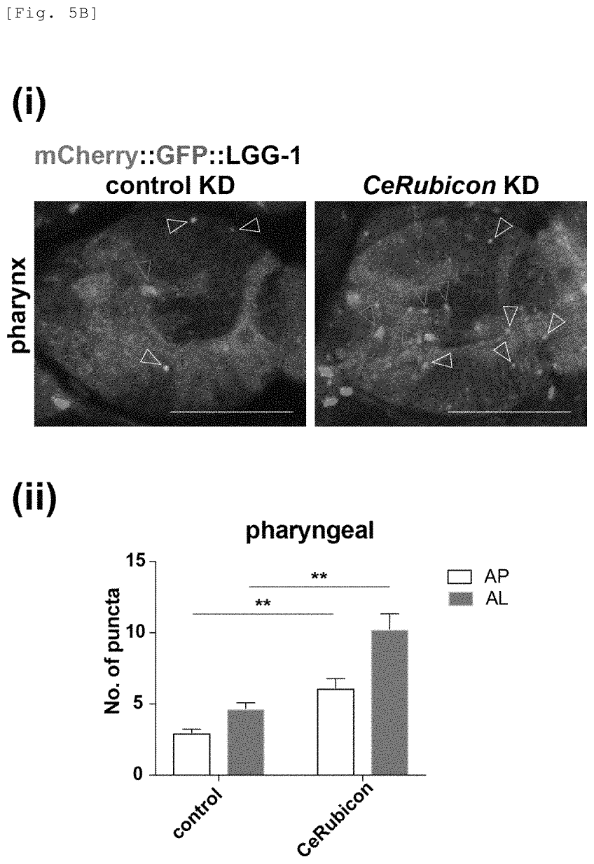

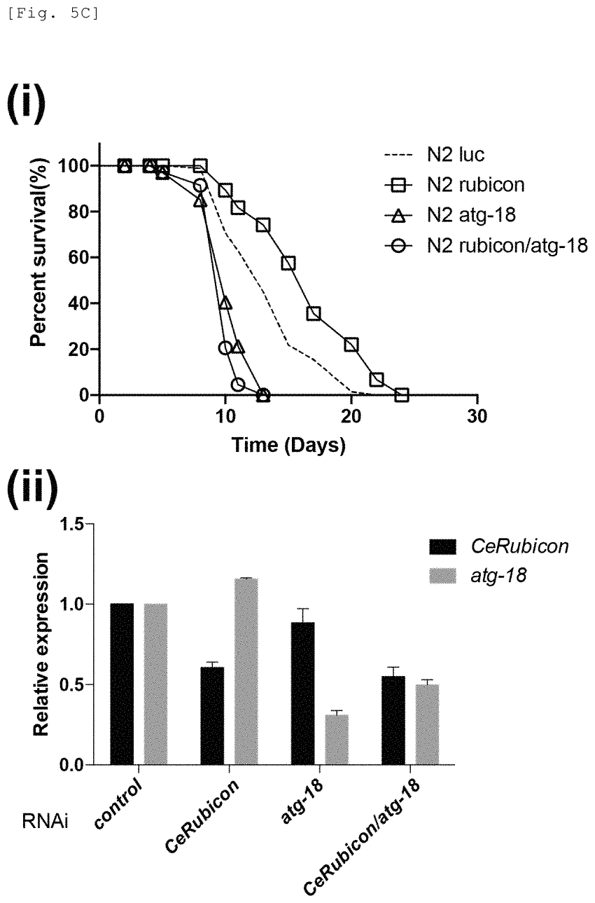

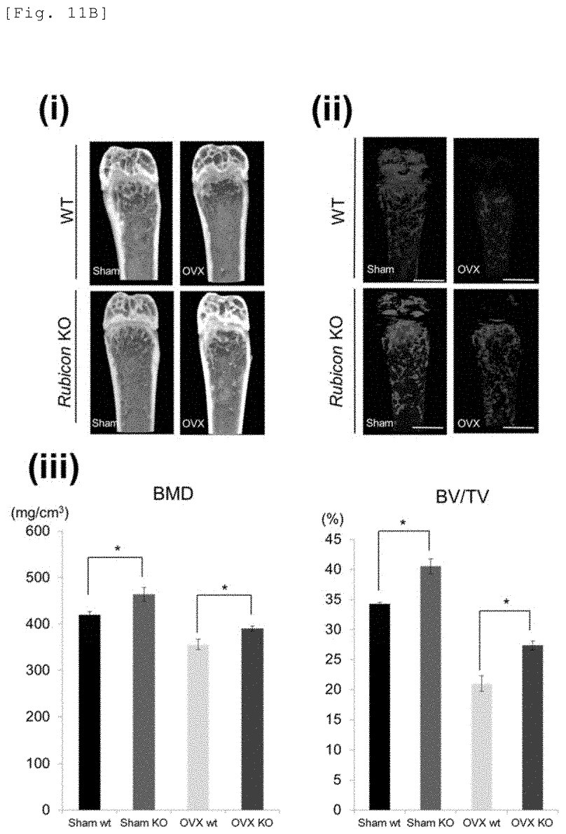

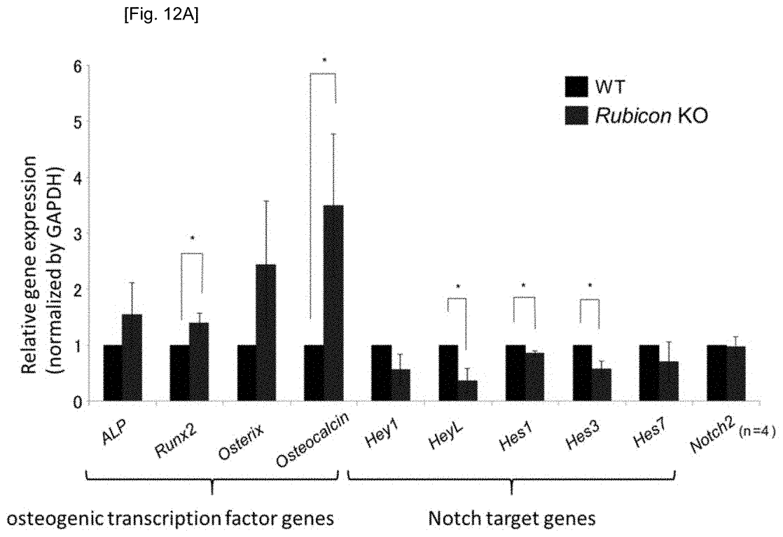

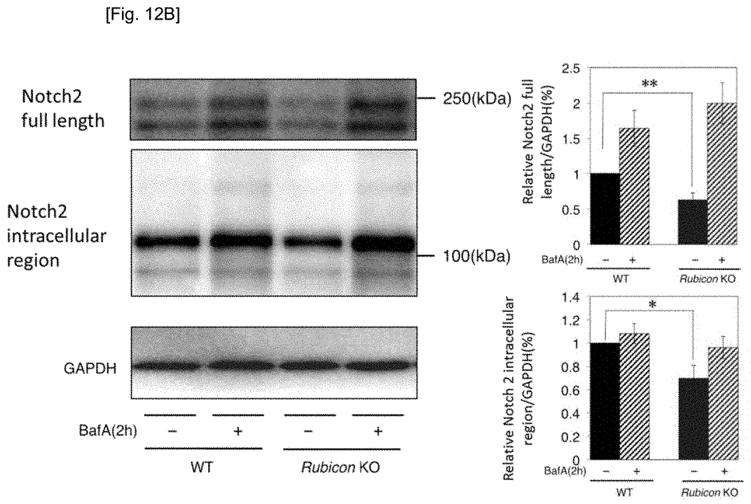

We also investigated which tissues of CeRubicon are responsible for lifespan regulation in C. elegans. We used tissue-specific, RNAi-sensitive strains, including TU3401 (neuron-specific, using the sid-1 system) (Calixto, A. et al., Nature Methods 7, 554-U102 (2010)), NR350 (muscle), VP303 (intestine), and NR222 (hypodermis) for CeRubicon knockdown ((i) in FIG. 3A, (i) to (iii) in FIG. 7A) (Qadota, H. et al., Gene 400, 166-173 (2007)). The knockdown of CeRubicon in neuronal cells extended the lifespans most efficiently ((i) in FIG. 3A). While lower than the above, hypodermal and intestinal knockdown of CeRubicon extended the lifespan to a lesser extent but significantly ((i) to (iii) in FIG. 7A). Because neuronal cells are normally resistant to RNAi knockdown, the extension of lifespans conferred by the CeRubicon knockdown in wild-type worms might reflect the combinational effect of hypodermis, intestine, and other unidentified tissues ((i), (ii) in FIG. 1D, (i) in FIG. 5C).

We next investigated whether increased autophagy activity in neurons by CeRubicon knockdown would be sufficient to extend animal lifespan. As a result, longevity enhancement conferred by CeRubicon knockdown in neuronal cells was completely abolished by concomitant knockdown of atg-18/WIPI ((i) in FIG. 7B). This suggests that the neuron activation of autophagy is sufficient to increase lifespan. Similarly, neuron-specific knockdown of Rubicon in fruit fly also extends lifespan in female fly ((ii) in FIG. 3A, (ii) in FIG. 7B). Moreover, we found that Rubicon knockdown decreased the polyQ inclusions in female brain neurons ((i), (ii) in FIG. 3B) and mitigated the age-associated decline in locomotor function, which is accelerated by the polyQ cytotoxicity ((iii) in FIG. 3B).

Autophagy is implicated in several age-associated neurodegenerative disorders, including Parkinson's disease. In sporadic and familial Parkinson's disease, affected neurons develop inclusions containing α-synuclein (α-Syn). Elevated amounts of this protein are sufficient to cause Parkinson's disease. Overexpressed and misfolded aggregate-prone forms of α-Syn are degraded by autophagy. Therefore, we investigated whether forced activation of autophagy by deletion of Rubicon in neurons would be sufficient to prevent aggregation of α-Syn in the brain. To explore this possibility, we injected preformed α-Syn fibrils into the striatum of control and Rubicon neuronal (Nestin-cre) knockout mice as previously reported (Luk, K. C. et al., Science 338, 949-953 (2012), Watanabe, Y. et al., Autophagy 13, 133-148 (2017)), and compared the formation of Lewy body and Lewy neurite-like α-Syn inclusions after 10 months. Remarkably, the level of phosphorylated α-Syn positive signals was significantly reduced in neuron-specific Rubicon knockout mice ((i), (ii) in FIG. 3C), suggesting that neuronal Rubicon deletion suppressed the expansion of α-synuclein pathology. Together, these results suggest that neuronal knockdown of Rubicon is sufficient to ameliorate aging phenotypes in multiple organisms.

(4) Downregulation of Rubicon in Several Lifespan-extending Conditions

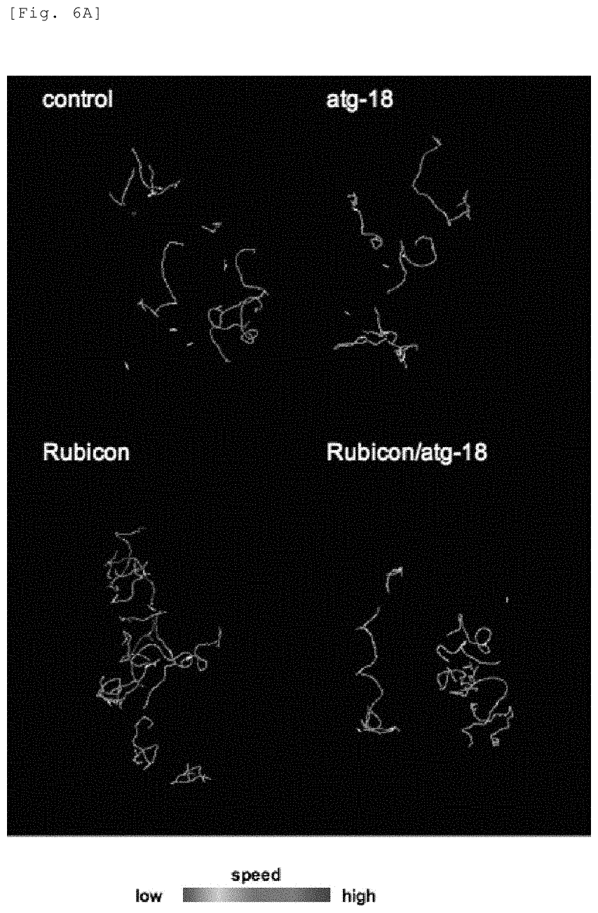

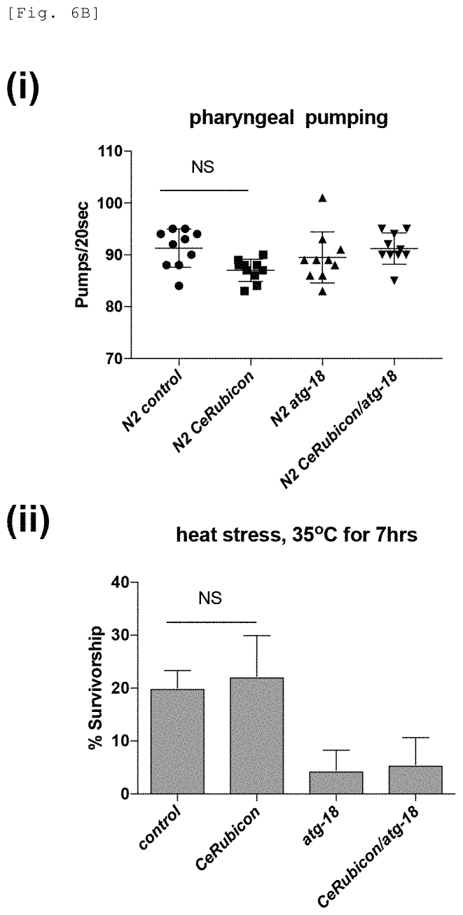

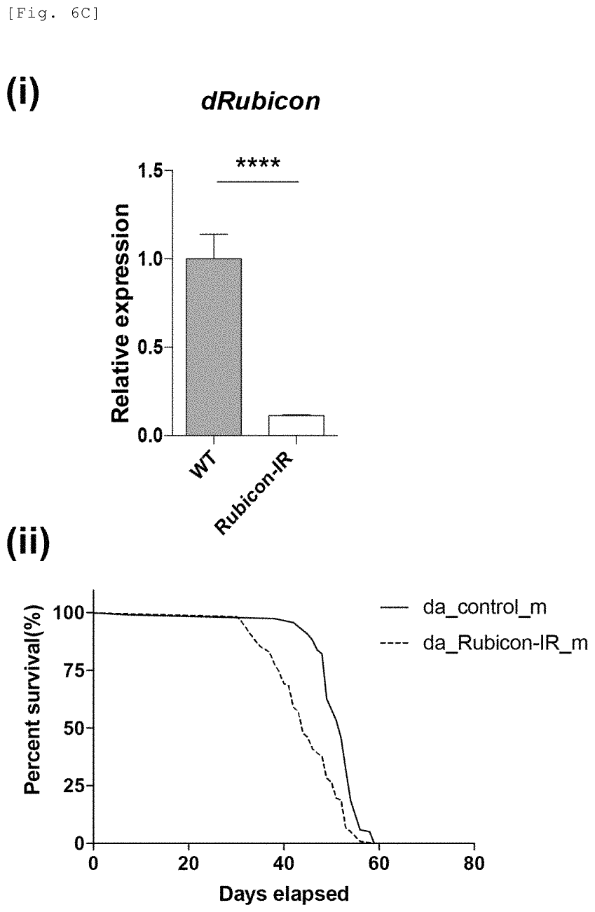

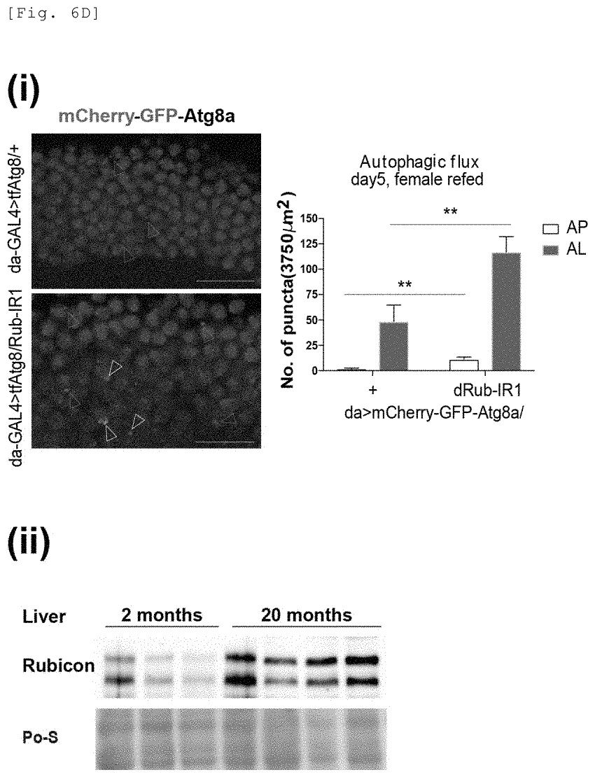

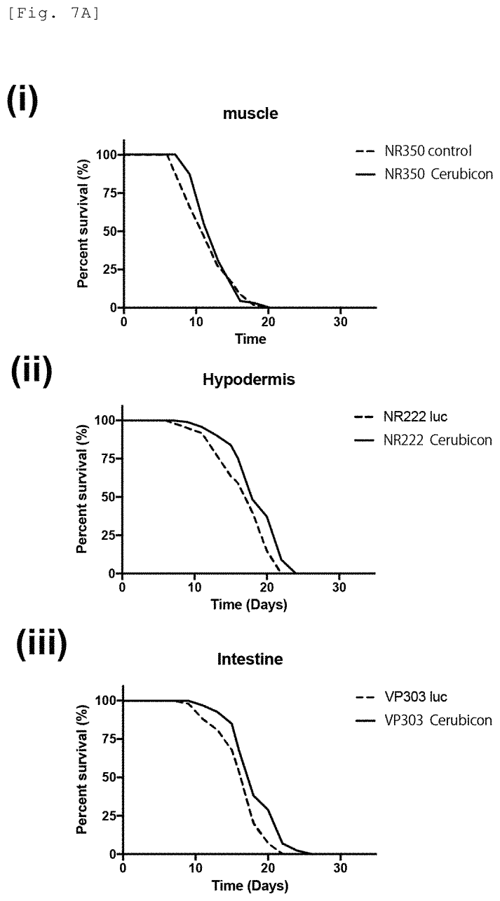

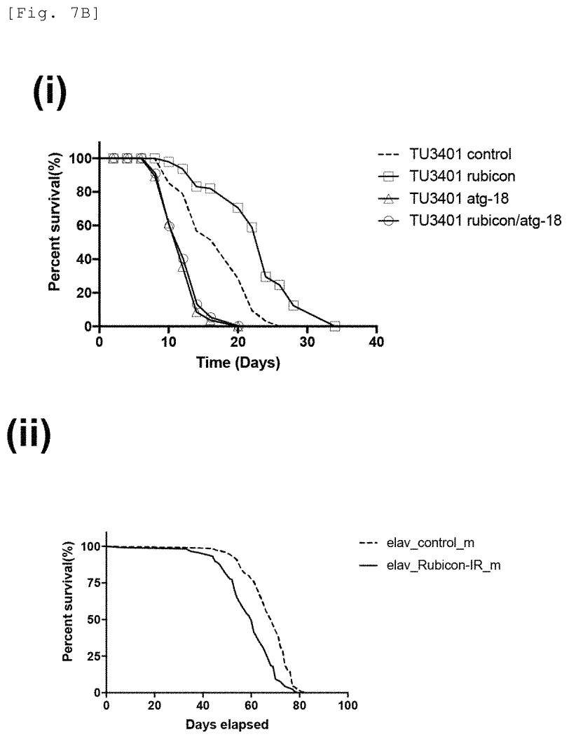

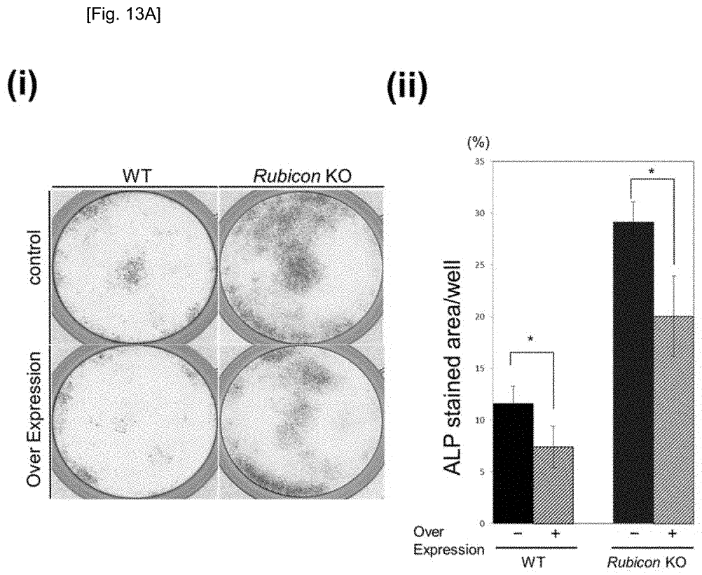

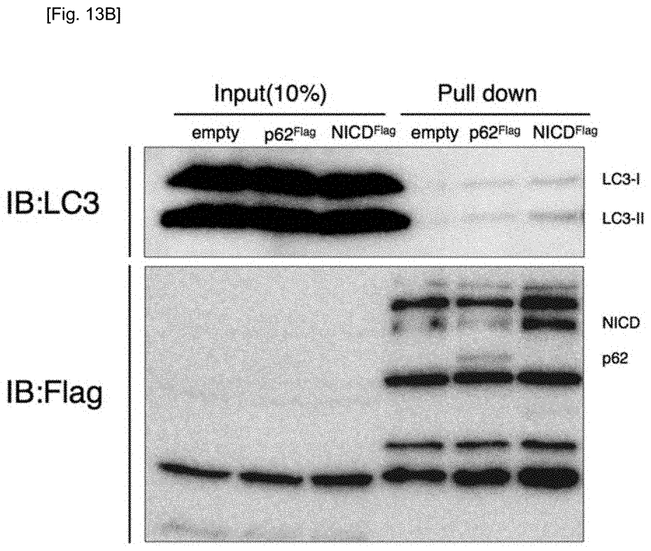

We investigated whether Rubicon expression is altered by several lifespan-extending conditions. In worms, several different longevity pathways and corresponding long-lived mutants have been well characterized. These include reduction of insulin/IGF-1 signalling (daf-2), germline removal (glp-1), calorie restriction (eat-2), and mitochondrial dysfunction (isp-1) (Kenyon, C. J. Nature 464, 504-512 (2010)). CeRubicon expression was suppressed in all of these long-lived worms in comparison with the wild-type ((i) in FIG. 4A, (ii) in FIG. 8B). Moreover, mice subjected to 9-month calorie restriction exhibited reduced Rubicon expression in liver and kidney ((ii) in FIG. 4A, (i) in FIG. 8B). In addition, we found that CeRubicon knockdown does not further extend longevity of daf-2, glp-1, eat-2, and isp-1 ((i) to (iv) in FIG. 8A). This result indicates that Rubicon is regulated downstream of multiple longevity paradigms, and the longevity of these animals is partly conferred by the reduction in Rubicon expression.

In worms, MML-1/Mondo has been identified as one of convergent transcription factors essential for multiple longevity pathways (Nakamura, S. et al., Nat Commun 7, 10944 (2016), Johnson, D. W. et al., Plos Genet 10 (2014)). In particular, knockdown of MML-1 abolishes autophagic activity and longevity in germline-deficient long-lived glp-1 animals. However, how MML-1 regulates autophagy is not fully characterized. Hence, we sought to address the possible involvement of MML-1 in regulation of CeRubicon. Interestingly, suppression of CeRubicon in glp-1 animals was completely reverted after knockdown of MML-1 or its heterodimeric partner, mxl-2 ((i) in FIG. 4B) although CeRubicon was still largely suppressed in other long-lived animals ((ii) in FIG. 8B). Moreover, MML-1 overexpression, which results in gradual lifespan extension (Nakamura, S. et al. described above), decreased CeRubicon expression in comparison with wild-type ((ii) in FIG. 4B). In addition, knockdown of MondoA, a mammal homolog of MML-1, increased Rubicon protein levels in mammalian cells ((i), (ii) in FIG. 4C) although the Rubicon transcription level was unchanged. These results indicate that Rubicon is regulated downstream of MML-1/MondoA especially in germline longevity context, and this regulation seems conserved in mammals.

The nutrient sensor mTOR is inactivated in response to nutrient deprivation, and inhibition of mTOR induces autophagy and extends the lifespan of a number of species (Kapahi, P. et al., Cell Metab 11, 453-465 (2010)). In addition, MML-1 regulates TOR activity by repressing the leucine sensor lars-1 (Nakamura, S. et al. described above). Hence, we examined the possible crosstalk between Rubicon and the mTOR pathway. As a result, surprisingly, we found that double knockdown of CeRubicon and let-363/mTOR further extended lifespan in comparison with either single knockdown (FIG. 4D). Knockdown of CeRubicon does not affect TOR activity, while TOR knockdown does not change CeRubicon expression ((i), (ii) in FIG. 8C). These results indicate that TOR and Rubicon, two major negative regulators of autophagy, contribute to longevity through partly independent downstream mechanisms.

(5) Adjustment of Bone Metabolism through Autophagy by Rubicon

We took images of the femurs of systemic Rubicon knockout mice with CT, and found that the bone mineral density (BMD), the bone volume/bone tissue volume fraction (BV/TV), and the bone mineral content (BMC) of the femurs were significantly increased in Rubicon knockout mice (FIG. 9A). Hence, we created primary culture systems of osteoclasts and osteoblasts, and evaluated the differentiation potential and function of these. We evaluated differentiation of osteoclasts through TRAP staining to find that the differentiation was facilitated in Rubicon knockout osteoclasts (FIG. 9B). In addition, we evaluated the bone resorption function of osteoclasts through pit formation assay to find that the bone resorption function was facilitated in Rubicon knockout osteoblasts (FIG. 9C). Next, we investigated the differentiation potential and the mineralization function of osteoblasts through the primary culture system and the culture cell system. Rubicon knockout cells in the culture cell system were formed with the Crisper-Cas9 system. We evaluated the differentiation potential through ALP staining to find that the differentiation potential was significantly increased in Rubicon knockout osteoblasts (FIG. 9D). In addition, we evaluated the mineralization function through alizarin staining to find that the mineralization function was facilitated in Rubicon knockout osteoblasts (FIG. 9E). In systemic Rubicon KO mice, differentiation potentials and functions were facilitated in both osteoclasts and osteoblasts, while in Rubicon knockout mice, the bone morphology of the femur was enhanced, leading us to assume that the function of osteoblasts is more important. In addition, we observed whether autophagy was facilitated in Rubicon knockout osteoblasts through LC3 flux assay. As a result, LC3 flux was facilitated both under nutrient condition and under starvation condition in Rubicon knockout osteoblasts (FIG. 10).

In forming osteoblast-specific mice, we crossed Osterix-cre mice and Rubicon^{flow/flox} mice to form Rubicon^{flow/flox};Osterix-Cre mice. At the same time, we crossed Osterix-cre mice with Atg5^{flox/flox} mice and Rubicon^{flow/flox}; Atg5^{flox/flox} mice to form osteoblast-specific Atg5 knockout mice and Rubicon/Atg5 knockout mice, and CT-analyzed their bone morphologies. As a result, osteoblast-specific Rubicon knockout mice exhibited significantly increased bone mineral density. On the other hand, it is reported that osteoblast-specific Atg7 knockout mice exhibit decreased bone mineral density in (Li, H. et al., Autophagy 14(10), 1726-1741 (2018)), and in this experiment as well, Atg5 knockout mice exhibited significantly decreased bone mineral density. In addition, Rubicon/Atg5 knockout mice exhibited bone mineral density decreased to a similar extent to Atg5 knockout mice (FIG. 11A). This fact indicates that osteogenesis in osteoblast-specific Rubicon knockout mice occurs in an autophagy-dependent manner.

In the present Examples, osteoblast-specific Rubicon knockout mice exhibited change in osteogenic ability, and we investigated whether this contributes to amelioration of osteoporosis by forming an osteoporotic model. As the osteoporotic model, we used a post-menopause osteoporotic model formed by extirpating both ovaries to deplete estrogen for 4 weeks. As a result, in sham mice, osteoporosis was induced, bone mineral density decreased, and bone volume/bone tissue volume fraction also decreased. On the other hand, in Rubicon knockout mice, bone mineral density and bone volume/bone tissue volume fraction were significantly ameliorated in the osteoporotic model (FIG. 11B).

Change in osteogenic ability was observed in an autophagy-dependent manner in Rubicon knockout mice, and autophagy, differentiation, and osteogenic function were facilitated in Rubicon knockout osteoblasts. This fact suggests that autophagy is involved in differentiation of osteoblasts. It has been known that the signal pathway of wnt, BMP, or Notch is involved in differentiation of osteoblasts (Zanotti, S. & Canalis, E. Molecular and Cellular Biology, 30(4), 886-896 (2010), Muguruma, Y. et al., Journal of Cellular Physiology, 232(9), 2569-2580 (2017)), in the present Examples, we focused on the signal pathway of Notch. This is because it has already been reported that osteoblast-specific Notch2 knockout mice (Notch has 1 to 4 isoforms, and in osteoblasts, Notch2 is highly expressed) exhibits change in osteogenic ability and differentiation and function of osteoblasts are also facilitated, and Notch receptor decreases in an autophagy-dependent manner (Yorgan, T. et al., Bone 87(C), 136-146 (2016), Wu, X. et al., Nature Communications 7, 1-17 (2016), Cao, Y. et al., Stem Cells and Development 24(22), 2660-2673 (2015)). In the previous report, the mechanism with which Notch2 knockout mice exhibit change in osteogenic ability of osteoblast has been reported as follows: specifically, deletion of Notch2 receptor lowers NICD which is transferred into nuclei to repress transcription factors downstream of Notch. Since Hes and Hey downstream of Notch negatively regulate Runx2 and Osterix, osteogenic transcription factors, decrease of Hes and Hey allows osteogenic transcription factors such as Runx2 and Osterix to increase, facilitating the differentiation potential and the osteogenesis function of osteoblasts. In view of this, the present inventors predicted that in Rubicon knockout osteoblasts, facilitated autophagy promotes degradation of the Notch receptor, so that Hes and Hey downstream of Notch decrease, and further Runx2 and Osterix increase. We observed expression of mRNAs of the transcription factor downstream of Notch and the osteogenic transcription factor, and found that the expression of mRNA decreased in the Rubicon knockout osteoblasts in Hey1, HeyL, Hes1, Hes3, and Hes7 downstream of Notch, whereas the expression of mRNA increased in the osteogenic transcription factors such as Runx2 and osteocalcin (FIG. 12A). Moreover, we observed whether Notch2 and NICD were degraded in Rubicon knockout osteoblasts through western blotting, and it was suggested that flux of Notch2 and NICD by lysosome inhibition increased in Rubicon knockout osteoblasts, and more Notch2 and NICD are degraded in Rubicon knockout cell (FIG. 12B).

Although degradation of Notch receptor and NICD was facilitated in Rubicon knockout osteoblasts, it was unclear whether NICD functioned downstream of Rubicon. In view of this, NICD was overexpressed in Rubicon knockout osteoblasts, and the differentiation potential of NICD was evaluated through ALP staining. The differentiation was repressed to a similar extent to wild-type osteoblasts. This fact revealed that NICD was regulated downstream of Rubicon (FIG. 13A). Since NICD has two LIR motifs, we considered a possibility that NICD served as an autophagic substrate. Hence, we used NICD added with Flag tag and observed the binding of NICD and LC3 by the immunoprecipitation method, in which immunoprecipitation was conducted with Flag, and LC3 was detected through immunoblotting. The result revealed that LC3 and NICD directly bind to each other (FIG. 13B).

### INDUSTRIAL APPLICABILITY

As described above, the present invention makes it possible to suppress aging associated with attenuation of autophagy with time, widely prevent, ameliorate, or treat an age-related disease or symptom, and extend lifespan, by targeting Rubicon. In addition, the present invention also makes it possible to test the degree of aging, the risk of developing these diseases or symptoms, or the risk of shortening lifespan, by targeting Rubicon. Therefore, the present invention greatly contributes particularly to the medical field.
[Sequence Listing] IBPF19-525WO-seq-fin.txt

1-11. (canceled)

12. A composition for suppression of aging, prevention, amelioration, or treatment of an age-related disease or symptom, or extension of lifespan in a subject, the composition comprising a molecule that suppresses functions of a Rubicon gene as an active ingredient or a compound that binds to a Rubicon protein.

13. The composition according to claim 12, wherein
the molecule that suppresses functions of a Rubicon gene is a molecule described in any one of the following (a) to (d):
(a) an RNA binding to a transcription product of the Rubicon gene or a DNA encoding the RNA,
(b) an RNA or DNA binding to the Rubicon protein,
(c) an antibody or low-molecular-weight compound binding to the Rubicon protein, and
(d) a site-specific nuclease for editing a nucleic acid sequence of the Rubicon gene or DNA of an expression regulating region thereof or the RNA sequence of a transcription product of the Rubicon gene.

14. The composition according to claim 12, wherein
the age-related disease or symptom is a metabolic bone disease or a symptom thereof, decrease in stress resistance, or an organ fibrosis.

15. The composition according to claim 12, wherein
the age-related disease or symptom is neurodegeneration or a symptom thereof, decrease in locomotor function, or eye degeneration.

16. The composition according to claim 12, wherein
the subject is a vertebrate.

17. A method for evaluating a probability that a test compound has an activity of suppressing aging, an activity of preventing, ameliorating, or treating an age-related disease or symptom, or an activity of extending lifespan in a subject, the method comprising the steps of:
bringing the test compound and a Rubicon protein into contact; and
detecting binding of the test compound and the Rubicon protein, wherein
in a case where the test compound binds to the Rubicon protein, it is estimated that there is the probability that the test compound has an activity of suppressing aging, an activity of preventing, ameliorating, or treating an age-related disease or symptom, or an activity of extending lifespan.

18. A method for evaluating a probability that a test compound has an activity of suppressing aging, an activity of preventing, ameliorating, or treating an age-related disease or symptom, or an activity of extending lifespan in a subject, the method comprising the steps of:
bringing the test compound into contact with a cell expressing a Rubicon gene; and
detecting expression of the Rubicon gene in the cell, wherein
in a case where the test compound reduces the expression of the Rubicon gene compared with a case where the test compound is not brought into contact, it is evaluated that there is the probability that the test compound has an activity of suppressing aging, an activity of preventing, ameliorating, or treating an age-related disease or symptom, or an activity of extending lifespan.

19. A method for evaluating a probability that a test compound has an activity of suppressing aging, an activity of preventing, ameliorating, or treating an age-related disease or symptom, or an activity of extending lifespan in a subject, the method comprising the steps of:
bringing the test compound into contact with a cell having a DNA in which a reporter gene is functionally bound to downstream of a promoter region of a Rubicon gene; and
detecting expression of the reporter gene in the cell, wherein
in a case where the test compound reduces the expression of the reporter gene compared with a case where the test compound is not brought into contact, it is evaluated that there is the probability that the test compound has an activity of suppressing aging, an activity of preventing, ameliorating, or treating an age-related disease or symptom, or an activity of extending lifespan.

20. A method for testing a degree of aging, a risk of developing an age-related disease or symptom, or a risk of shortening lifespan in a subject, the method comprising the step of:
detecting an expression product of a Rubicon gene in a test sample, wherein
in a case where an amount of the expression product of the Rubicon gene is higher than that of control, it is evaluated that the degree of aging, the risk of developing an age-related disease or symptom, or the risk of shortening lifespan is high.

21. The method according to claim 17, wherein
the age-related disease or symptom is a metabolic bone disease or a symptom thereof, decrease in stress resistance, or an organ fibrosis.

22. The method according to claim 18, wherein
the age-related disease or symptom is a metabolic bone disease or a symptom thereof, decrease in stress resistance, or an organ fibrosis.

23. The method according to claim 17, wherein
the age-related disease or symptom is neurodegeneration or a symptom thereof, decrease in locomotor function, or eye degeneration.

24. A composition for detecting an expression product of a Rubicon gene in the method according to claim 20, the composition comprising the following (a) or (b):
(a) an oligonucleotide primer or oligonucleotide probe binding to a transcription product of the Rubicon gene; or
(b) an antibody binding to a Rubicon protein.

25. The composition according to claim 14, wherein
the age-related disease or symptom is osteoporosis, decrease in bone mass, decrease in bone mineral density and bone volume/bone tissue volume fraction, or change in bone morphology.

26. The composition according to claim 15, wherein
the age-related disease or symptom is Parkinson's disease, Alzheimer's disease, polyglutamine disease or amyotrophic lateral sclerosis.

27. The method according to claim 21, wherein
the age-related disease or symptom is osteoporosis, decrease in bone mass, decrease in bone mineral density and bone volume/bone tissue volume fraction, or change in bone morphology.

28. The method according to claim 23, wherein
the age-related disease or symptom is Parkinson's disease, Alzheimer's disease, polyglutamine disease or amyotrophic lateral sclerosis.

29. A composition targeting Rubicon for suppression of aging, prevention, amelioration, or treatment of an age-related disease or symptom, or extension of lifespan in a subject.

30. The method according to claim 19, wherein
the age-related disease or symptom is a metabolic bone disease or a symptom thereof, decrease in stress resistance, or an organ fibrosis.

31. The method according to claim 20, wherein
the age-related disease or symptom is a metabolic bone disease or a symptom thereof, decrease in stress resistance, or an organ fibrosis.

32. The method according to claim 18, wherein
the age-related disease or symptom is neurodegeneration or a symptom thereof, decrease in locomotor function, or eye degeneration.

33. The method according to claim 19, wherein
the age-related disease or symptom is neurodegeneration or a symptom thereof, decrease in locomotor function, or eye degeneration.

34. The method according to claim 20, wherein
the age-related disease or symptom is neurodegeneration or a symptom thereof, decrease in locomotor function, or eye degeneration.

35. The method according to claim 22, wherein
the age-related disease or symptom is osteoporosis, decrease in bone mass, decrease in bone mineral density and bone volume/bone tissue volume fraction, or change in bone morphology.

36. The method according to claim 30, wherein
the age-related disease or symptom is osteoporosis, decrease in bone mass, decrease in bone mineral density and bone volume/bone tissue volume fraction, or change in bone morphology.

37. The method according to claim 31, wherein
the age-related disease or symptom is osteoporosis, decrease in bone mass, decrease in bone mineral density and bone volume/bone tissue volume fraction, or change in bone morphology.

38. The method according to claim 32, wherein
the age-related disease or symptom is Parkinson's disease, Alzheimer's disease, polyglutamine disease or amyotrophic lateral sclerosis.

39. The method according to claim 33, wherein
the age-related disease or symptom is Parkinson's disease, Alzheimer's disease, polyglutamine disease or amyotrophic lateral sclerosis.

40. The method according to claim 34, wherein
the age-related disease or symptom is Parkinson's disease, Alzheimer's disease, polyglutamine disease or amyotrophic lateral sclerosis.
